(19) United States
(12) Patent Application Publication
(10) Pub. No.: US 2021/0087191 A1
(43) Pub. Date: Mar. 25, 2021

(54) SUBSTITUTED 3-ISOBUTYL-9,10-DIMETHOXY-1,3,4,6,7,11B-HEXAHYDRO-2H-PYRIDO[2,1-a]ISOQUINOLIN-2-OL COMPOUNDS, THEIR SYNTHESIS, AND USE THEREOF

(71) Applicant: DISPERSOL TECHNOLOGIES, LLC, Georgetown, TX (US)

(72) Inventors: Dave Alan MILLER, Georgetown, TX (US); Daniel J. ELLENBERGER, Cedar Park, TX (US); Esmir GUNIC, San Diego, CA (US); Jean-Luc GIRARDET, Poway, CA (US); Angela SPANGENBERG, Austin, TX (US)

(21) Appl. No.: 17/111,569

(22) Filed: Dec. 4, 2020

(51) Int. Cl. C07D 471/04; C07D 519/00

(52) U.S. Cl. CPC C07D 471/04; C07D 519/00

(57) ABSTRACT

The invention relates to substituted 3-isobutyl-9,10-dimethoxy-1,3,4,6,7,11b-hexahydro-2H-pyrido[2,1-a]isoquinolin-2-ol compounds, their synthesis, pharmaceutical compositions containing them, and methods of using them in the treatment of disorders benefiting from inhibition of vesicular monoamine transporter 2 (VMAT2).

### TECHNICAL FIELD

This invention relates to substituted 3-isobutyl-9,10-dimethoxy-1,3,4,6,7,11B-hexahydro-2H-pyrido[2,1-a]isoquinolin-2-ol compounds, and methods of making and administering such compounds. The invention also relates to pharmaceutical compositions containing such compounds, methods of making and administering such pharmaceutical compositions, and methods of treatment using such pharmaceutical compositions.

### BACKGROUND OF THE INVENTION

3-isobutyl-9,10-dimethoxy-1,3,4,6,7,11b-hexahydro-2H-pyrido[2,1-a]isoquinolin-2-one, also known as tetrabenazine (TBZ), is a potent, reversible inhibitor of catecholamine uptake by vesicular monoamine transporter-2 (VMAT2) (IC₅₀=3.2 nM). Inhibition of VMAT2 by TBZ results in a reversible depletion of brain monoamines in vivo (such as dopamine, serotonin, norepinephrine, histamine) from nerve terminals.

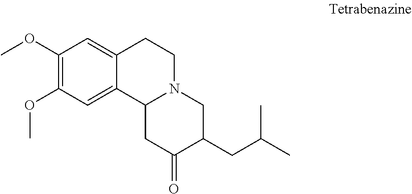 [SMILES: COc1cc2c(cc1OC)C1CC(=O)C(CC(C)C)CN1CC2]

In use as a pharmaceutical drug since the late 1950s, initially as an anti-psychotic, TBZ is currently used for treating hyperkinetic movement disorders such as Huntington's disease, hemiballismus, senile chorea, tic, tardive dyskinesia, and Tourette's syndrome, and is FDA approved to treat chorea associated with Huntington's disease. However, significant dose limiting side-effects are associated with the use of TBZ. Specifically, the dose must be titrated for each individual patient via a prolonged dose escalation process. Escalation to the most effective dose is limited by the onset of side-effects, i.e., dose titration is stopped once side effects are experienced. This process introduces a safety concern in the eliciting of side effects in each patient and limits the efficacy of the therapy in that the optimum dose may not be achieved prior to the onset of side effects. There is evidence to support that the side effects associated with TBZ are related to the metabolism of TBZ to metabolites (see below) that have off-target interactions and/or reduced efficacy (Grigoriadis et al., J. Pharmacol. Exp. Ther. 361:454-461 (2017)).

The most common adverse reactions include sedation, somnolence, suicidal thoughts, akathisia, neuroleptic malignant syndrome, drowsiness, fatigue, nervousness, anxiety, insomnia, agitation, confusion, orthostatic hypotension, nausea, dizziness, depression, and Parkinsonism. There is a black-box warning associated with the use of TBZ for increased risk of depression and suicidal thoughts and behavior in patients with Huntington's disease.

The current commercial TBZ tablet formulation is a standard immediate release (IR) composition designed for rapid and complete release of TBZ in the gastric environment. Human PK profiles obtained after oral administration of a 25 mg IR formulation have been disclosed in e.g., Roberts et al., Eur. J. Clin. Pharmacol. 29:703-708 (1986) and Derangula et al., Biomed Chromatogr. 27:792-801 (2013). The oral PK is characterized by a high peak plasma concentration followed by a steep decline in plasma concentration. Mean Tₘₐₓ is approximately 1 hour and mean t_{1/2} is approximately 2 hours.

TBZ is a weakly basic compound with relatively good solubility in acidic environments (8.5 mg/mL at pH<2) and poor solubility in neutral environments (0.03 mg/mL at pH>4). The solubility of the compound in the gastric environment leads to near-complete absorption from the IR composition following oral administration because the compound is rapidly and completely dissolved and absorbed. Conversely, for a controlled release formulation, the composition passes through the gastric environment allowing for only minimal dissolution of the compound. Consequently, much of the dose is transitioned to the intestinal tract where the pH of the lumen is not conducive to the dissolution of the compound. For example, the pH in the duodenum is about 5-7 and increases to about 7-8 in the ileum and decreases slightly in the colon to 5-7 (Gruber et al., Adv. Drug. Del. Reviews 1:1-18 (1987); Evans et al., Gut 29:1035-141 (1988)). At these pH levels TBZ is practically insoluble. As a result, incomplete absorption is achieved from the intestinal tract, systemic concentrations remain low, and efficacy is compromised.

TBZ contains two chiral centers (*) at the 3 and 11b carbon atoms, as shown below

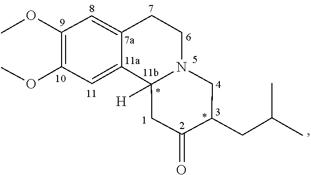 [SMILES: COc1cc2c(cc1OC)C1CC(=O)C(CC(C)C)CN1CC2]

and can, theoretically, exist in a total of four isomeric forms, as shown below (the stereochemistry of each isomer defined using the “R” and “S” nomenclature)

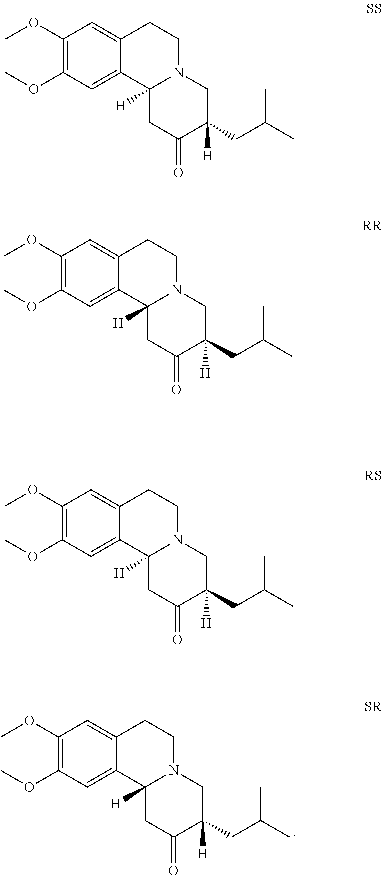

TBZ is rapidly and extensively metabolized in vivo to its reduced form, 1,3,4,6,7,11b-hexahydro-9,10-dimethoxy-3-(2-methylpropyl)-2H-benzo[a]quinolizin-2-ol, also known as dihydrotetrabenazine (DHTBZ).

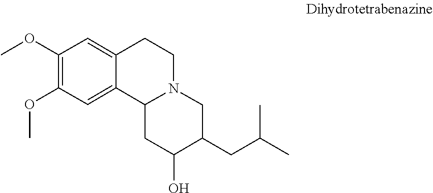 [SMILES: COc1cc2c(cc1OC)C1CC(O)C(CC(C)C)CN1CC2]

DHTBZ has three chiral centers at the 2, 3, and 11b carbon atoms, and can therefore exist as one of the following eight separate isomers

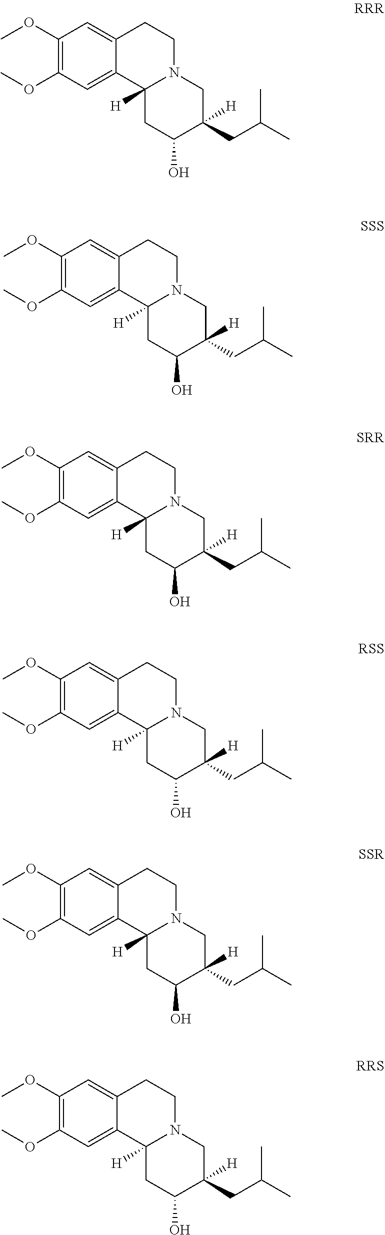

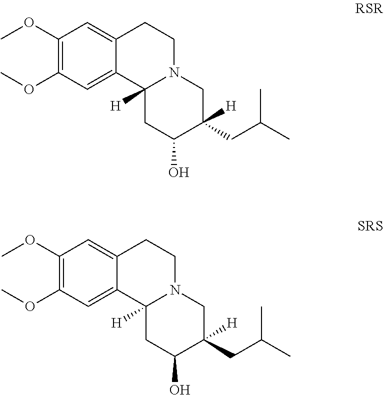

Of the eight DHTBZ isomers, four isomers are derived from the more stable RR and SS isomers of the parent TBZ, namely the RRR, SSS, SRR and RSS isomers.

The RRR and SSS isomers are commonly referred to as “alpha (α)” DHTBZs and can be referred to individually as (+)-α-DHTBZ and (−)-α-DHTBZ respectively. The alpha isomers are characterized by a trans relative orientation of the hydroxyl and 2-methylpropyl substituents at the 2- and 3-positions.

The SRR and RSS isomers are commonly referred to as “beta (ß)” isomers and can be referred to individually as (+)-ß-DHTBZ and (−)-ß-DHTBZ respectively. The beta isomers are characterized by a cis relative orientation of the hydroxyl and 2-methylpropyl substituents at the 2- and 3-positions.

(+)-α-DHTBZ is believed to be the absolute configuration of the active metabolite (Kilbourn et al., Chirality 9:59-62 (1997)). Grigoriadis et al. determined that (+)-α-DHTBZ has the lowest inhibitory concentration for VMAT2 and has no off-target affinity, compared to the other three isomers (Grigoriadis et al., J. Pharmacol. Exp. Ther. 361:454-461 (2017)).

As discussed above, it is known that TBZ exhibits several dose-related side effects including causing depression and parkinsonism (see WO2016/127133). It appears that these side-effects may also be caused by VMAT2 inhibition and that consequently it is difficult to separate the therapeutic effect of TBZ and TBZ-derived compounds from these side-effects (Grigoriadis et al., J. Pharmacol. Exp. Ther. 361:454-461 (2017)).

To avoid or reduce the side-effects associated with TBZ, a valine ester prodrug of (+)-α-DHTBZ has been developed, known by its INN name, Valbenazine. Valbenazine contains three chiral centers (in addition to the L-Valine group), (*) as shown below. It is administered therapeutically as (2R,3R,11bR)-9,10-dimethoxy-3-(2-methylpropyl)-1,3,4,6,7,11b-hexahydro-2H-benzo[a]quinolizin-2-yl L-valinate.

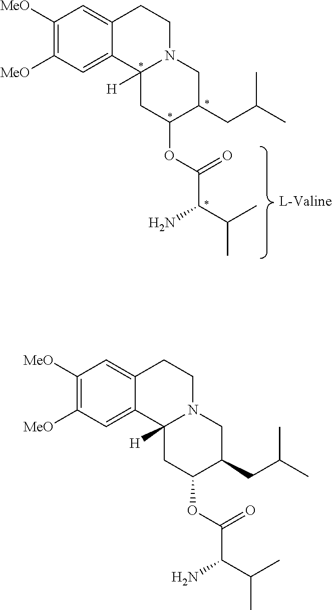 [SMILES: COc1cc2c(cc1OC)C1CC(OC(=O)[C@@H](N)C(C)C)C(CC(C)C)CN1CC2 ; COc1cc2c(cc1OC)[C@H]1C[C@@H](OC(=O)[C@@H](N)C(C)C)[C@H](CC(C)C)CN1CC2]

Valbenazine and its metabolite (+)-α-DHTBZ cause reversible reduction of dopamine release at the nerve terminal by selectively inhibiting the pre-synaptic human VMAT2.

In vitro, valbenazine is a selective inhibitor of human VMAT2 showing little or no affinity for VMAT1, other receptors (e.g., dopamine D2 receptors), transporters, and ion channels.

Valbenazine has an oral bioavailability of 49%, t_{1/2}=15-22 hours, and a Tₘₐₓ of 0.5-1 h. Approximately 60% of Valbenazine is excreted in urine and 30% in feces. Less than 2% of the parent compound or active metabolite is excreted unchanged. Valbenazine is extensively metabolized in vivo, via two mechanisms:

(i) hydrolysis of the valine ester by CYP3A4/5, to form (+)-α-DHTBZ (Cₘₐₓ 4-8 hours); and

(ii) oxidation (primarily by CYP3A4/5) to form mono-oxidized valbenazine.

Due to its relatively slow metabolism, high doses (40 mg or 80 mg) of Valbenazine, compared to TBZ, are required for the treatment of adults with tardive dyskinesia to maintain circulating levels, sufficient to hit target.

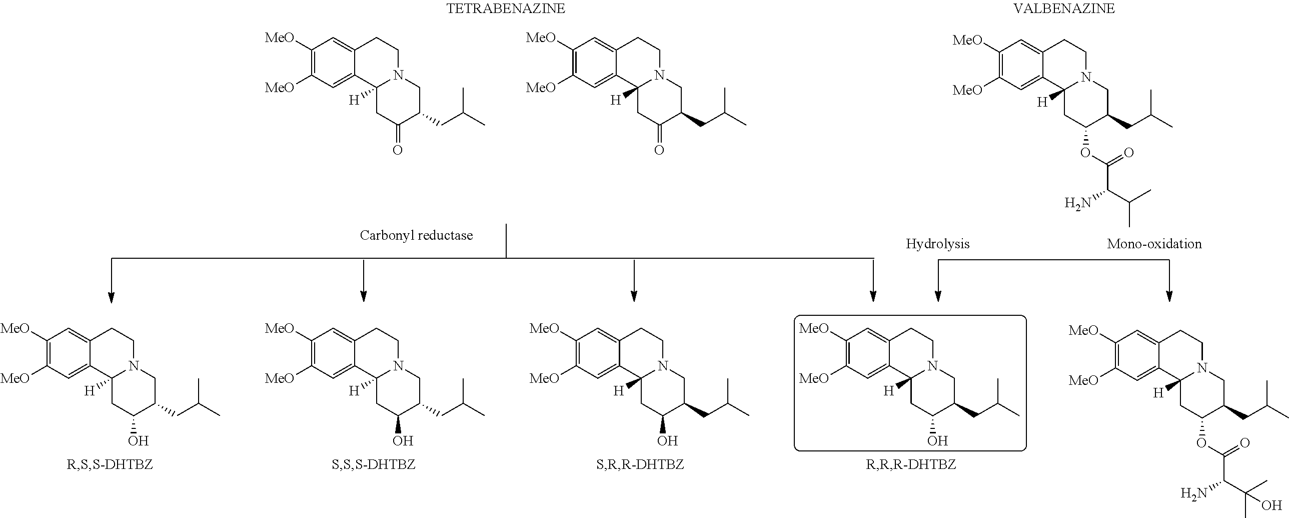

Valbenazine is FDA approved for the treatment of adults with Tardive Dyskinesia (TD) and has been used in clinical trials to study the treatment of Tourette Syndrome. However, it has a warning for side-effects including somnolence (including impaired ability to drive or operate hazardous machinery) and QT Prolongation and may cause an increase in QT interval, (patients with congenital long QT syndrome or with arrhythmias associated with a prolonged QT interval should avoid Valbenazine). Furthermore, because (+)-α-DHTBZ Tₘₐₓ is not reached until 4-8 hours after the administration of Valbenazine, Valbenazine is inadequate for immediate inhibition of VMAT2.

Movement disorders, such as tardive dyskinesia, can be triggered by anxiety, agitation, and stress (Gerlach et al., Acta psychiatr. Scand. 77:369-378 (1988)). At present, there are no viable treatments for sudden dyskinesia. As discussed, Ingrezza® (Valbenazine) is not rapid acting and has a median half-life of the active metabolite of 4-8 hours (Kim, Drugs 77:1123-1129 (2017)). Austedo® (Deutetrabenazine) is not rapid acting and has a median Tₘₐₓ of 3-4 hours (NDA 208082 (<<https://www.accessdat.fda.gov/drugsatfda_docs/nda/2017/208082Orig1s000ClinPharmR.pdf>>)). Xenazine® (Tetrabenazine) has a Tₘₐₓ of 1 hour and is dose limited by onset of side effects (NDA 208082 (<<https://www.accessdat.fda.gov/drugsatfda_docs/nda/2017/208082Orig1s000ClinPharmR.pdf>>)).

There is a need for a new immediate-release formulation of (+)-α-DHTBZ with an improved PK profile that can deliver very rapid blood concentrations of a VMAT2 inhibitor without the side effects that come from other TBZ metabolites. Particularly, there is a need for a new immediate-release formulation of (+)-α-DHTBZ which produces a PK profile with a lower Tₘₐₓ compared with existing immediate-release formulations of (+)-α-DHTBZ. There is also a need for an immediate-release formulation of (+)-α-DHTBZ which provides a higher Cₘₐₓ, Cₗₐₛₜ, and/or Area Under the Dissolution Curve (AUDC) than other immediate-release formulations of (+)-α-DHTBZ. Moreover, there is a need for slower-release formulations, such as those that produce a PK profile with a Tₘₐₓ of greater than 1.5 hours (e.g., 3-4, 4-8, 8-24 hours, etc.). Finally, there is a need for an immediate-release and slower-release formulations of (+)-α-DHTBZ which can treat VMAT2-mediated disorders.

Compounds and pharmaceutical compositions of the invention may avoid one or more of these side-effects or adverse reactions, and have improved physical properties.

### SUMMARY OF THE INVENTION

The invention relates to a compound of Formula (I):

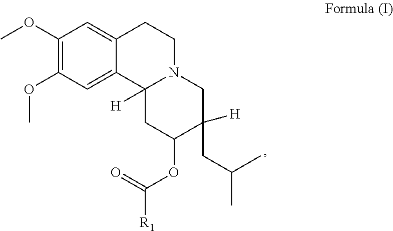

wherein:
R₁ is —C(O)R₂, —(C₁-C₆alkyl)-C(O)R₂, —(C₂-C₄alkenyl)-C(O)R₂, —NH—(C₁-C₄alkyl), —O—CH₂—O—C(O)—(C₁-C₄alkyl), or

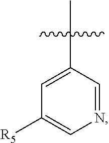

R₂ is —OR₃ or —NH—CH(—C(O)OH)—R₄,
R₃ is —H or —C₁-C₄alkyl,
R₄ is —C₁-C₄alkyl, optionally substituted by a phenyl group, and
R₅ is —C(O)OR₃,
or a pharmaceutically acceptable salt, ester, hydrate, or solvate thereof.

The invention also relates to a compound of Formula (II):

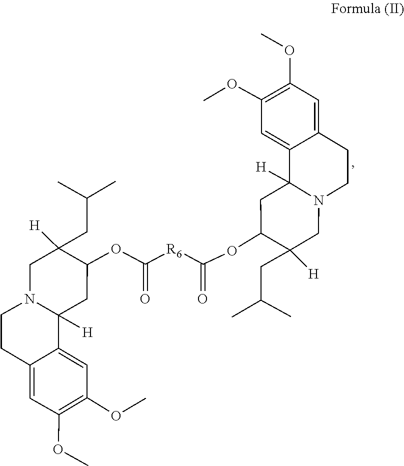

wherein:
R₆ is a bond, —C₁-C₈alkyl-, —C₂-C₄alkenyl-, or

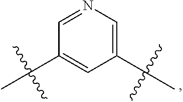 [SMILES: *c1cncc(*)c1]

or a pharmaceutically acceptable salt, ester, hydrate, or solvate thereof.

The invention further relates to a pharmaceutical composition comprising, consisting essentially of, or consisting of a therapeutically effective amount of a compound of Formula (I) or (II), and at least one pharmaceutically acceptable excipient.

The invention also relates to a method of administering the pharmaceutical composition of the invention to a mammal in the oral mucosa thereof, comprising:

a) providing the pharmaceutical composition; and

b) administrating the pharmaceutical composition to the mammal via a buccal route, a sublingual route, or a gingival route.

The invention also relates to a method of treating a VMAT2-mediated disorder to a mammal in need thereof, comprising administering the pharmaceutical composition of the invention.

### DESCRIPTION OF THE INVENTION

The invention relates to substituted 3-isobutyl-9,10-dimethoxy-1,3,4,6,7,11b-hexahydro-2H-pyrido[2,1-a]isoquinolin-2-ol compounds of Formula (I) or Formula (II), as well as stereoisomers, pharmaceutically acceptable salts, solvates, polymorphs, esters, and tautomers thereof.

The compound of Formula (I) of this invention has the following structure:

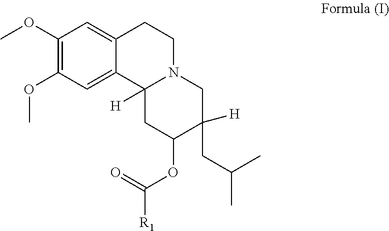

wherein:
R₁ is —C(O)R₂, —(C₁-C₆alkyl)-C(O)R₂, —(C₂-C₄alkenyl)-C(O)R₂, —NH—(C₁-C₄alkyl), —O—CH₂—O—C(O)—(C₁-C₄alkyl), or

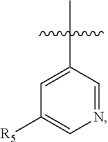

R₂ is —OR₃ or —NH—CH(—C(O)OH)—R₄,
R₃ is —H or —C₁-C₄alkyl,
R₄ is —C₁-C₄alkyl, optionally substituted by a phenyl group, and
R₅ is —C(O)OR₃.

Preferably, in the compound of Formula (I), R₁ is —C(O)R₂, —CH₂CH₂—C(O)R₂, —(CH₂)₆—C(O)R₂, —CH═CH—C(O)R₂, —NH—CH₂CH₃, —NH—CH(CH₃)₂, —NH—CH₂CH(CH₃)₂, —O—CH₂—O—C(O)—CH₂CH₃, —O—CH₂—O—C(O)—CH(CH₃)₂, —O—CH₂—O—C(O)—CH₂CH(CH₃)₂, or

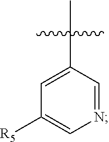

R₂ is —OR₃ or —NH—CH(—C(O)OH)—R₄; R₃ is —H or —CH(CH₃)₂; R₄ is —CH(CH₃)₂ or —CH₂(phenyl); and R₅ is —C(O)OR₃.

The compound of Formula (I) may exist as any one of the following enantiomeric forms:

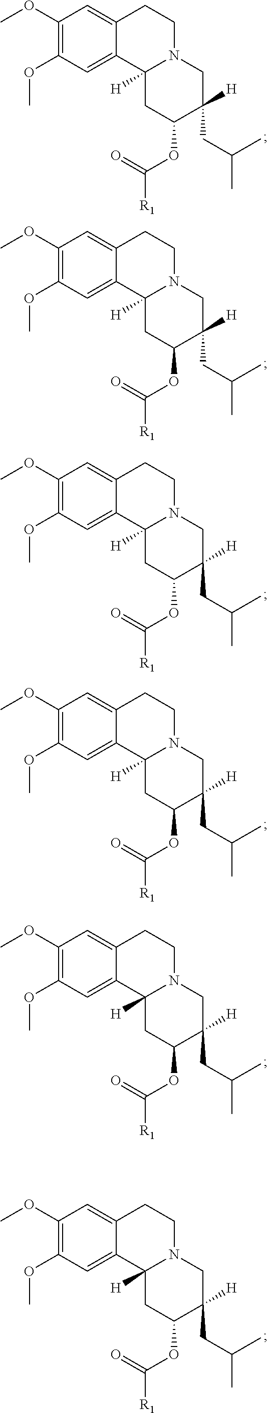

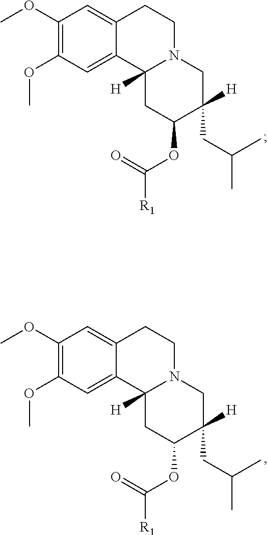

wherein R₁-R₅ are as defined herein.

Preferably, the compound of Formula (I) has the following enantiomeric structure:

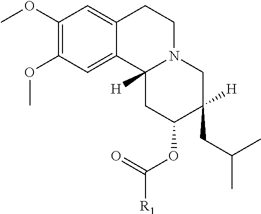

wherein R₁-R₅ are as defined herein.

Preferred compounds of Formula (I) include the following:
(2S,3S,11bS)-9,10-dimethoxy-3-(2-methylpropyl)-1H,2H,3H,4H,6H,7H,11bH-pyrido[2,1-a]isoquinolin-2-yl hydrogen oxalate;
4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid;
(2E)-4-[[(2S,3S,11bS)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid;
(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid;
8-[[(2R,3R,11bR)-9,10-dimethoxy-3-(2-methylpropyl)-1H,2H,3H,4H,6H,7H,11bH-pyrido[2,1-a]isoquinolin-2-yl]oxy]-8-oxooctanoic acid;
5-([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)pyridine-3-carboxylic acid;
(2R,3R,11bR)-9,10-dimethoxy-3-(2-methylpropyl)-1H,2H,3H,4H,6H,7H,11bH-pyrido[2,1-a]isoquinolin-2-yl 1-isopropyl butanedioate;
(2R,3R,11bR)-9,10-dimethoxy-3-(2-methylpropyl)-1H,2H,3H,4H,6H,7H,11bH-pyrido[2,1-a]isoquinolin-2-yl 1-isopropyl (2E)-but-2-enedioate;
3-((2R,3R,11bR)-3-isobutyl-9,10-dimethoxy-1,3,4,6,7,11b-hexahydro-2H-pyrido[2,1-a]isoquinolin-2-yl) 5-isopropyl pyridine-3,5-dicarboxylate;
(2R,3R,11bR)-9,10-dimethoxy-3-(2-methylpropyl)-1H,2H,3H,4H,6H,7H,11bH-pyrido[2,1-a]isoquinolin-2-yl 1-isopropyl (2Z)-but-2-enedioate;
(2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid;
(2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid;
(2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid;
(2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid;
(2R,3R,11bR)-9,10-dimethoxy-3-(2-methylpropyl)-1H,2H,3H,4H,6H,7H,11bH-pyrido[2,1-a]isoquinolin-2-yl N-ethylcarbamate;
(2R,3R,11bR)-9,10-dimethoxy-3-(2-methylpropyl)-1H,2H,3H,4H,6H,7H,11bH-pyrido[2,1-a]isoquinolin-2-yl N-isopropyl carbamate;
(2R,3R,11bR)-9,10-dimethoxy-3-(2-methylpropyl)-1H,2H,3H,4H,6H,7H,11bH-pyrido[2,1-a]isoquinolin-2-yl N-(2-methylpropyl)carbamate;
[([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl propanoate;
[([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 2-methylpropanoate; and
[([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 3-methylbutanoate.

The compound of Formula (II) of this invention has the following structure:

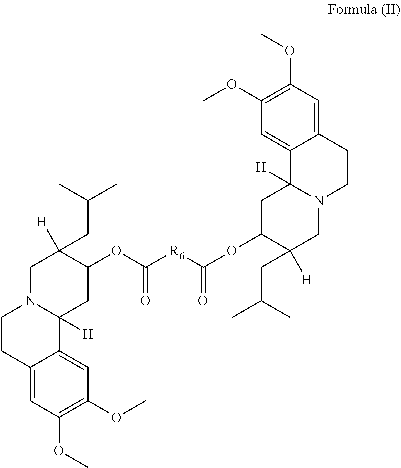

wherein:
R₆ is a bond, —C₁-C₈alkyl-, —C₂-C₄alkenyl-, or

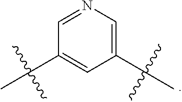 [SMILES: *c1cncc(*)c1]

Preferably, in the compound of Formula (II), R₆ is a bond, —CH₂CH₂—, —(CH₂)₆—, —CH═CH—, or

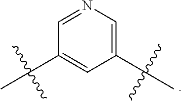 [SMILES: *c1cncc(*)c1]

The compound of Formula (II) may exist as any one of its enantiomeric forms. Preferably, the compound of Formula (II) has the following enantiomeric structure:

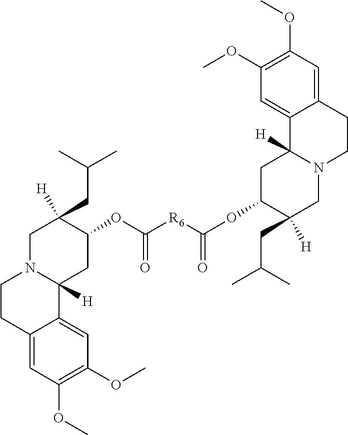

wherein R₆ is as defined herein.

Preferred compounds of Formula (II) include the following:
3,5-bis[(2R,3R,11bR)-9,10-dimethoxy-3-(2-methylpropyl)-1H,2H,3H,4H,6H,7H,11bH-pyrido[2,1-a]isoquinolin-2-yl] pyridine-3,5-dicarboxylate;
bis[(2R,3R,11bR)-9,10-dimethoxy-3-(2-methylpropyl)-1H,2H,3H,4H,6H,7H,11bH-pyrido[2,1-a]isoquinolin-2-yl] octanedioate;
[(2R,3R,11bR)-9,10-dimethoxy-3-(2-methylpropyl)-1H,2H,3H,4H,6H,7H,11bH-pyrido[2,1-a]isoquinolin-2-yl] butanedioate;
bis[(2R,3R,11bR)-9,10-dimethoxy-3-(2-methylpropyl)-1H,2H,3H,4H,6H,7H,11bH-pyrido[2,1-a]isoquinolin-2-yl] (2E)-but-2-enedioate;
bis[(2R,3R,11bR)-9,10-dimethoxy-3-(2-methylpropyl)-1H,2H,3H,4H,6H,7H,11bH-pyrido[2,1-a]isoquinolin-2-yl] (2Z)-but-2-enedioate; and
bis[(2R,3R,11bR)-9,10-dimethoxy-3-(2-methylpropyl)-1H,2H,3H,4H,6H,7H,11bH-pyrido[2,1-a]isoquinolin-2-yl] oxalate.

As used herein, the term “alkyl” refers to a linear, branched, saturated hydrocarbon group, such as methyl, ethyl, n-propyl, iso-propyl, n-butyl, iso-butyl, tert-butyl, pentyl, hexyl, and the like.

As used herein, the term “alkenyl” refers to a linear, branched hydrocarbon group containing at least one double bond, such as ethenyl, n-propenyl, iso-propenyl, n-butenyl, iso-butenyl, pentenyl, hexenyl, and the like.

The compounds of Formula (I) and (II) may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹¹C, ¹²D, ¹³D, and ¹⁴C and ¹⁶O and ¹⁸O.

Typically, the compounds of Formula (I) and (II) do not contain isotopes (such as ¹¹O or ³H) in amounts higher than their natural occurrence.

Preferably, the percentage of the total hydrogen atoms in the compounds of Formula (I) and (II) that are deuterium atoms is less than 2%, more typically less than 1%, more usually less than 0.1%, preferably less than 0.05%, and most preferably no more than 0.02%. Most preferred, the compounds of Formula (I) and (II) have no deuterium atoms.

The isotopes may be radioactive or non-radioactive. Preferably, the compounds of Formula (I) and (II) contain no radioactive isotopes. Such compounds are preferred for therapeutic use. However, the compounds of Formula (I) and (II) may contain one or more radioisotopes. The compounds of Formula (I) and (II) containing such radioisotopes may be useful in a diagnostic context.

The compounds of Formula (I) and (II) typically have an isomeric purity of greater than 60%. The term “isomeric purity” means the amount of (+)-α-DHTBZ free base present in the compounds of Formula (I) and (II) relative to the total amount or concentration of DHTBZ of all isomeric forms. For example, if 90% of the total DHTBZ present in the composition is (+)-α-DHTBZ, then the isomeric purity is 90%. The compounds of Formula (I) and (II) of the invention may have an isomeric purity of greater than 82%, greater than 85%, greater than 87%, greater than 90%, greater than 91%, greater than 92%, greater than 93%, greater than 94%, greater than 95%, greater than 96%, greater than 97%, greater than 98%, greater than 99%, greater than 99.5%, or greater than 99.9%.

The compounds of Formula (I) and (II) will generally be administered to a subject in need of such administration, for example a human or animal patient, preferably a human.

The compounds of Formula (I) and (II) will typically be administered in amounts that are therapeutically or prophylactically useful and which generally are non-toxic. However, in certain situations, the benefits of administering a compound of Formula (I) or (II) may outweigh the disadvantages of any toxic effects or side effects, in which case it may be considered desirable to administer compounds in amounts that are associated with a degree of toxicity.

The compounds of Formula (I) and (II) is effective in the treatment of VMAT2-mediated disorders at much lower doses than could have been predicted from the literature (e.g., from WO 2015/171802) and that its use at such lower doses can avoid or minimize the unwanted side effects associated with TBZ.

Pharmaceutical Compositions and Methods of Treatment

The invention also relates to a pharmaceutical composition comprising, consisting essentially of, or consisting of a therapeutically effective amount of at least one compound of Formula (I) or (II) and at least one pharmaceutically acceptable excipient.

Preferably, the pharmaceutical composition contains between about 0.1% wt. % and about 99.9 wt. %, such as between about 0.5 wt. % and about 99.5 wt. %, such as between about 1 wt. % and about 99 wt. %, of the at least one compound of Formula (I) or (II). The total amount of compound of Formula (I) or (II) in the pharmaceutical compositions of the invention may range from about 0.0001-500 mg, about 0.0001-100 mg, about 0.001-50 mg, about 0.01-25 mg, about 0.1-5 mg, about 1-4 mg, about 1-3 mg, or about 1-2 mg. For example, the pharmaceutical compositions of the invention may contain 0.0001 mg, 0.001 mg, 0.01 mg, 0.1 mg, 0.5 mg, 1 mg, 1.5 mg, 2 mg, 2.5 mg, 3 mg, 3.5 mg, 4 mg, 4.5 mg, or 5 mg of the compound of Formula (I) or (II).

The at least one pharmaceutically acceptable excipient may be an effervescent agent, a pH adjusting substance, a surfactant, a processing aid (e.g., plasticizers), a permeation enhancer, a pharmaceutical polymer, a disintegrant, a filler, a lubricant (e.g., stearates), a preservative (e.g., parabens), a glidant, a binder, an antioxidant, a thickener, a sweetener, a flavorant, a coloring component, and mixtures thereof. Preferably, the pharmaceutical composition contains between about 0.1 wt. % and about 99.9 wt. %, such as between about 0.5 wt. % and about 99.5 wt. %, such as between about 1 wt. % and about 99 wt. %, of the at least one the pharmaceutically acceptable excipient.

Preferably, the pharmaceutically acceptable excipient is an effervescent agent and/or a pH adjusting substance. The effervescent agent may be, for example, sodium bicarbonate, sodium carbonate, potassium bicarbonate and potassium carbonate, magnesium carbonate, and mixtures thereof. The effervescent agent may be oral fluid activated. The effervescent agent may be present in an amount sufficient to increase absorption of the compound in the oral mucosa. The effervescent agent may be present in an amount sufficient to evolve a gas in an amount between about 5 cm³ to about 30 cm³ upon exposure to oral fluid. The effervescent agent is present in an amount between about 5 wt. % and about 95 wt. %, such as about 30 wt. % and about 80 wt. %, of the dosage form.

The at least one pH adjusting substance may be an acidifying agent (e.g., a carboxylic acid), alkalizing agent, and a buffering agent. The acidifying agents may be, for example, adipic acid, ammonium chloride, citric acid monohydrate, lactic acid, and tartaric acid. The alkalizing agents may be, for example, calcium hydroxide, magnesium carbonate, potassium carbonate, potassium bicarbonate, potassium citrate, potassium hydroxide, sodium carbonate, sodium bicarbonate, sodium borate, sodium citrate dihydrate, and sodium hydroxide. And the buffering agents may be, for example, adipic acid, boric acid, calcium carbonate, calcium hydroxide, calcium lactate, calcium phosphate tribasic, citric acid monohydrate, dibasic sodium phosphate, glycine, maleic acid, malic acid, methionine, monobasic sodium phosphate, monosodium glutamate, potassium citrate, sodium acetate, sodium bicarbonate, sodium borate, sodium carbonate, sodium citrate dihydrate, sodium hydroxide, and sodium lactate. The at least one pH adjusting substance is present in an amount sufficient to adjust the pH in the oral fluid. The at least one pH adjusting substance is selected and provided in an amount capable of providing a change in localized pH in the oral fluid of at least 0.5 pH units. The at least one pH adjusting substance may be present in an amount between about 0.5 wt. % to about 25 wt. %, such as about 2 wt. % to about 20 wt. %, of the dosage form.

The at least one pH adjusting substance may also not be a component of the at least one effervescent agent.

Examples of the surfactant include sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, and sorbitan laurate.

Examples of the permeation enhancer include bile salts, fatty acids and derivatives, glycerides, chelators, and salicylates.

Examples of the pharmaceutical polymer include poly(vinylpyrrolidone), hydroxypropylcellulose, poly(vinyl alcohol), hydroxypropyl methylcellulose, hydroxyethylcellulose, and sodium carboxymethyl-cellulose, and polyvinyl caprolactam-polyvinyl acetate-polyethylene glycol graft copolymer. Water-soluble polymers may be used as well, such as poly(vinyl acetate)-co-poly(vinylpyrrolidone) copolymer, poly(vinylpyrrolidone), cellulose acetate phthalate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropyl methylcellulose, hydroxypropylmethylcellulose acetate succinate, poly(vinyl alcohol), and polyvinyl caprolactam-polyvinyl acetate-polyethylene glycol graft copolymer.

Examples of the disintegrant include sodium starch glycolate, crospovidone, magnesium aluminum silicate, microcrystalline cellulose, croscarmellose sodium, and cross-linked hydroxypropyl cellulose. The at least one disintegrant may be present in an amount sufficient to cause the dosage form to completely disintegrate in the oral fluid within 10 minutes, such as 5 minutes, 2 minutes, or 1 minute, from contacting the oral fluid. The at least one disintegrant may be present in an amount between about 0.25 wt. % to about 20 wt. %, such as about 2 wt. % to about 10 wt. %, of the dosage form.

Examples of the filler include mannitol, dextrose, lactose, sucrose, calcium carbonate, sorbitol, xylitol, and glucose. The at least one filler may be present in an amount between about 10 wt. % and about 80 wt. % of the dosage form.

The compound of Formula (I) or (II) may also not be substantially encompassed by or dispersed in a material in the dosage form that prevents absorption of the compound in the oral mucosa.

The pharmaceutical composition may also comprise at least one additional active pharmaceutical ingredient.

The pharmaceutical composition may exist as a unit dosage form, such as, for example, a solid oral dosage form, and, as such, will typically contain an amount of the compound of Formula (I) or (II) to provide a desired level of biological activity. The compound of Formula (I) or (II) will be administered to a subject (patient) in need thereof (e.g., a human or animal patient) in an amount sufficient to achieve the desired therapeutic effect. The solid oral dosage form may be, for example, a tablet, an oral tablet, a sublingual tablet, an oral capsule, a film, a buccal tablet, a buccal patch, or a polymer strip.

The oral unit dosage form may further include at least one bioadhesive, wherein the bioadhesive increases the contact time between the dosage form and the oral mucosa. The at least one bioadhesive may be, for example, an alginate, a lectin, a carageenan, a pectin, a cellulosic material, and mixtures thereof.

Preferably, the oral unit dosage form may disintegrate in oral fluid (e.g., saliva) and may be substantially absorbed in the oral mucosa. The oral dosage form may substantially disintegrate in the oral fluid within 10 minutes, such as within 2 minutes, such as within 1 minute from contacting the oral fluid. Alternatively, the oral unit dosage form may disintegrate in the stomach and may be substantially absorbed in the gastrointestinal tract.

The pharmaceutical compositions of the invention can be formulated in accordance with known techniques, see for example, Remington's Pharmaceutical Sciences, Mack Publishing Company, Easton, Pa., USA.

The invention also relates to a method of administering the pharmaceutical composition of the invention to a mammal, such as a human, in the oral mucosa thereof, comprising, consisting of, or consisting essentially of:
a) providing the pharmaceutical composition; and
b) administrating the pharmaceutical composition to the mammal via a buccal route, a sublingual route, or a gingival route.

The pharmaceutical composition may be administered such that it provides a Tₘₐₓ of 1.5 hours or less, such as 1.0 hours or less. Alternatively, the pharmaceutical composition may be administered such that it provide a Tₘₐₓ of greater than 1.5 hours, such as 3-4, 4-8, 8-24 hours, etc.

The pharmaceutical composition may be administered such that it provides a ratio of Cₘₐₓ to dose of between about 2.0 and about 4.0 picograms/mL/microgram.

The pharmaceutical composition may also be administered such that it provides a (+)-α-DTHBZ Cₘₐₓ of between about 75% to about 125%, such as between about 80% and about 120%, such as between about 85% to about 115%, that of a Xenazine® formulation wherein the latter includes at least 80% more 2R,3R,11bR-dihydrotetrabenazine by weight.

The invention also relates to the use of the pharmaceutical composition of the invention to treat a VMAT2-mediated disorder. The VMAT2-mediated disorder may be chronic hyperkinetic disorder, Huntington's disease, hemiballismus, senile chorea, tic disorders, tardive dyskinesia, levodopa-induced dyskinesia (LID), dystonia, Tourette's syndrome, depression, cancer, rheumatoid arthritis, psychosis, multiple sclerosis, asthma, Parkinson's disease, drug addiction, environmental neurotoxin-mediated neurodegeneration, bipolar disorder, and schizophrenia. Preferably, the pharmaceutical compositions of the invention may be used to treat LID, including acute onset of LID symptoms.

The invention also provides at least one compound of Formula (I) or (II) for use in medicine; as a VMAT2 receptor antagonist; and in the treatment of a VMAT2-mediated disorder.

The invention also relates to a method of treatment of a VMAT2-mediated disorder in a subject in need thereof (e.g., a mammalian subject such as a human), which method comprises administering to the subject a therapeutically effective amount of at least one compound of Formula (I) or (II).

The invention further relates to the use of at least one compound of Formula (I) or (II) for the manufacture of a medicament for the treatment of a VMAT2-mediated disorder.

The terms “treat,” “treating,” and “treatment” as used herein pertains generally to treatment and therapy in which some desired therapeutic effect is achieved, for example, the inhibition of the progress of the condition, and includes a reduction in the rate of progress, a halt in the rate of progress, amelioration of the condition, diminishment or alleviation of at least one symptom associated or caused by the condition being treated and cure of the condition. When the hyperkinetic movement disorder being treated is Tourette's Syndrome, for example, treatment of the disorder may pertain to a reduction of the incidence or severity of tics.

The quantity of the at least one compound of Formula (I) or (II) specified may be administered once per day or in several (e.g., two, three, four, five, etc.) doses per day.

The administration of the at least one compound of Formula (I) or (II) typically forms part of a chronic treatment regime. The at least one compound of Formula (I) or (II) may therefore be administered to a patient for a treatment period of at least a week, more usually at least two weeks, or at least a month, and typically longer than a month. Where a patient is shown to respond well to treatment, the period of treatment can be longer than six months and may extend over a period of years.

The chronic treatment regime may involve the administration of the at least one compound of Formula (I) or (II) every day, or the treatment regime may include days when no compound of Formula (I) or (II) is administered.

The dosage administered to the subject may vary during the treatment period. For example, the initial dosage may be increased or decreased depending on the subject's response to the treatment. A subject may, for example, be given an initial low dose to test the subject's tolerance towards the at least one compound of Formula (I) or (II), and the dosage thereafter increased as necessary. Alternatively, an initial daily dosage administered to the patient may be selected so as to give an estimated desired degree of VMAT2 blockage, following which a lower maintenance dose may be given for the remainder of the treatment period, with the option of increasing the dosage should the subject's response to the treatment indicate that an increase is necessary.

### EXPERIMENTAL

### Example 1: (2S,3S,11bS)-9,10-dimethoxy-3-(2-methylpropyl)-1H,2H,3H,4H,6H,7H,11bH-pyrido[2,1-a]isoquinolin-2-yl hydrogen oxalate

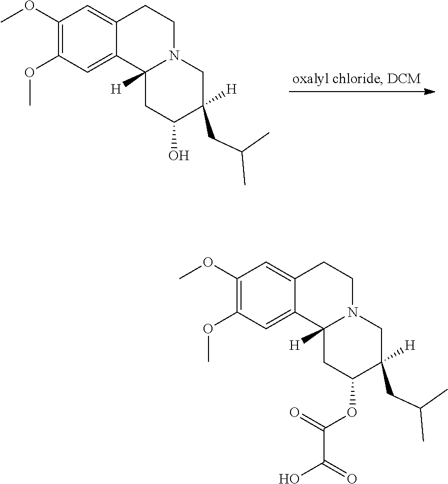

To a mixture of (2S,3S,11bS)-9,10-dimethoxy-3-(2-methylpropyl)-1H,2H,3H,4H,6H,7H,11bH-pyrido[2,1-a]isoquinolin-2-ol (400 mg, 1.252 mmol, 1 equiv) in DCM (2 mL) was added oxalyl chloride (1.90 mL, 2 mol/L, 3 equiv) at 0° C. The resulting solution was stirred at room temperature for 1 h. The reaction was then quenched by the addition of water. The resulting mixture was extracted with 2×15 mL of dichloromethane. The organic layers were combined and concentrated under vacuum. The residue was purified by Prep-HPLC with MeCN/H₂=32/68. This resulted in 137.0 mg (27.95%) of (2S,3S,11bS)-9,10-dimethoxy-3-(2-methylpropyl)-1H,2H,3H,4H,6H,7H,11bH-pyrido[2,1-a]isoquinolin-2-yl hydrogen oxalate as a white solid.

LCMS (ESI): [M+H]⁺: 392.3

¹H NMR (300 MHz, Methanol-d₄) δ 6.83 (s, 2H), 5.02-4.97 (m, 1H), 4.46-4.42 (m, 1H), 3.84 (s, 3H), 3.82 (s, 3H), 3.78-3.66 (m, 2H), 3.34-3.32 (m, 1H), 3.33-3.32 (m, 1H), 3.09-2.96 (m, 3H), 2.36-2.19 (m, 1H), 1.92-1.88 (m, 1H), 1.88-1.70 (m, 1H), 1.57-1.49 (m, 1H), 1.23-1.15 (m, 1H), 1.01-0.95 (m, 6H).

### Example 2: 4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid

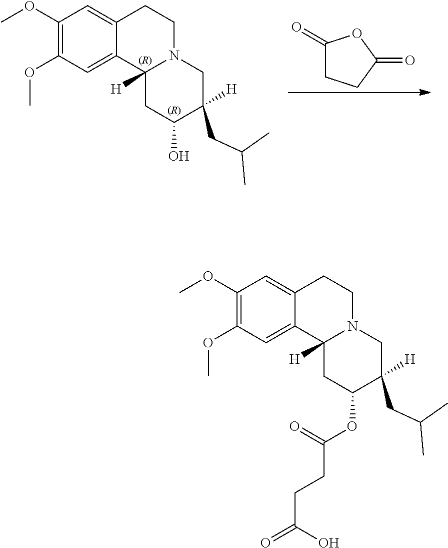

To a stirred mixture of (2R,3R,11bR)-9,10-dimethoxy-3-(2-methylpropyl)-1H,2H,3H,4H, 6H,7H,11bH-pyrido[2,1-a]isoquinolin-2-ol (400 mg, 1.252 mmol, 1 equiv) in DCM (5 mL) was added succinic anhydride (250 mg, 2.498 mmol, 2 equiv) and DMAP (306 mg, 2.505 mmol, 2 equiv) at room temperature. The resulting mixture was stirred at room temperature for 14 h. The resulting mixture was concentrated under vacuum. The residue was purified by Prep-HPLC with following conditions: Column: Xselect CSH F-Phenyl OBD column, 19*250, 5 um; Mobile Phase A: Water (0.1% FA), Mobile Phase B: ACN; Flow rate: 25 mL/min; Gradient: 5 B to 16 B in 10 min; 254/220 nm. This resulted in 144.8 mg (27.56%) 4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid as a light yellow solid.

LCMS (ESI): [M+H]⁺: 420.2

¹H NMR (300 MHz, Methanol-d₄) δ 6.75-6.73 (s, 1H), 6.73-6.72 (s, 1H), 4.86-4.73 (m, 1H), 3.80-3.51 (s, 6H), 3.48-3.36 (m, 1H), 3.33-3.11 (m, 3H), 2.79-2.70 (m, 3H), 2.70-2.57 (m, 4H), 2.35-2.27 (m, 1H), 2.13-1.95 (m, 1H), 1.82-1.61 (m, 1H), 1.55-1.51 (m, 1H), 1.51-1.38 (m, 1H), 1.12-1.07 (m, 1H), 1.07-0.92 (m, 6H).

### Example 3: (2E)-4-[[(2S,3S,11bS)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid

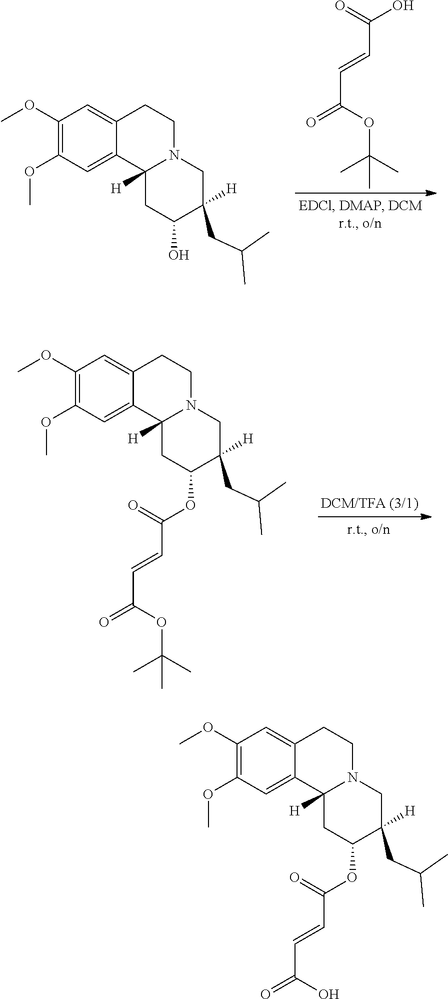

### (2R,3R,11bR)-9,10-dimethoxy-3-(2-methylpropyl)-1H,2H,3H,4H,6H,7H,11bH-pyrido[2,1-a]isoquinolin-2-yl 1-tert-butyl (2E)-but-2-enedioate

To a mixture of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) in DCM (20 mL) were added (2E)-4-(tert-butoxy)-4-oxobut-2-enoic acid (404 mg, 2.348 mmol, 1.5 equiv), EDCl (900 mg, 4.696 mmol, 3 equiv) and DMAP (191 mg, 1.565 mmol, 1 equiv). The resulting solution was stirred at room temperature overnight. The reaction was concentrated. The residue was applied onto a silica gel column with ethyl acetate/petroleum ether (1/2). This resulted in 370 mg (49.91%) of (2R,3R,11bR)-9,10-dimethoxy-3-(2-methylpropyl)-1H,2H,3H,4H,6H,7H,11bH-pyrido[2,1-a]isoquinolin-2-yl 1-tert-butyl (2E)-but-2-enedioate as a yellow solid.

LCMS (ESI): [M+H]⁺: 474.5

### (2E)-4-[[(2S,3S,11bS)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid

To a mixture of (2R,3R,11bR)-9,10-dimethoxy-3-(2-methylpropyl)-1H,2H,3H,4H,6H,7H,11bH-pyrido[2,1-a]isoquinolin-2-yl 1-tert-butyl (2E)-but-2-enedioate (350 mg, 0.739 mmol, 1 equiv) in DCM (3 mL) was added TFA (1 mL). The resulting solution was stirred at room temperature overnight. The resulting mixture was concentrated. The crude product was purified by Flash-Prep-HPLC with MeCN/H₂O=27/73. This resulted in 189.8 mg (55.41%) of (2E)-4-[[(2S,3S,11bS)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid as a light yellow solid.

LCMS (ESI): [M+H]⁺: 418.2

¹H NMR (400 MHz, Methanol-d₄) δ 6.94 (d, J=15.8 Hz, 1H), 6.81 (s, 1H), 6.80 (s, 1H), 6.63 (d, J=15.8 Hz, 1H), 5.00-4.98 (m, 1H), 4.18-4.15 (m, 1H), 3.83-3.81 (m, 6H), 3.61-3.52 (m, 2H), 3.23-3.14 (m, 2H), 3.05-2.88 (m, 3H), 2.16-2.30 (m, 1H), 1.88-1.74 (m, 2H), 1.41-1.38 (m, 1H), 1.21-1.18 (m, 1H), 0.98-0.94 (m, 6H).

### Example 4: (2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid

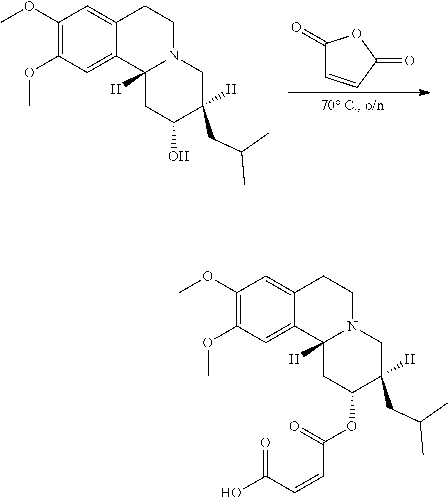

A mixture of furan-2,5-dione (1.5 g, 15.603 mmol, 10 equiv) and (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) was stirred at 70° C. overnight. The resulting mixture was concentrated. The crude product was purified by Prep-HPLC with MeCN/H₂O=32/68. This resulted in 101.2 mg (15.49%) of (2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid as a white solid.

LCMS (ESI): [M+H]⁺: 418.3

¹H NMR (300 MHz, Methanol-d₄) δ 6.80 (s, 1H), 6.79 (s, 1H), 6.59 (d, J=12.1 Hz, 1H), 5.88 (d, J=12.1 Hz, 1H), 4.95-4.91 (m, 1H), 4.16-4.10 (m, 1H), 3.82 (s, 6H), 3.60-3.53 (m, 2H), 3.31-3.13 (m, 2H), 2.98-2.81 (m, 3H), 2.31-2.06 (m, 1H), 1.88-1.60 (m, 2H), 1.53-1.45 (m, 1H), 1.17-1.11 (m, 1H), 0.96-0.93 (m, 6H).

### Example 5: 8-[[(2R,3R,11bR)-9,10-dimethoxy-3-(2-methylpropyl)-1H,2H,3H,4H,6H,7H,11bH-pyrido[2,1-a]isoquinolin-2-yl]oxy]-8-oxooctanoic acid

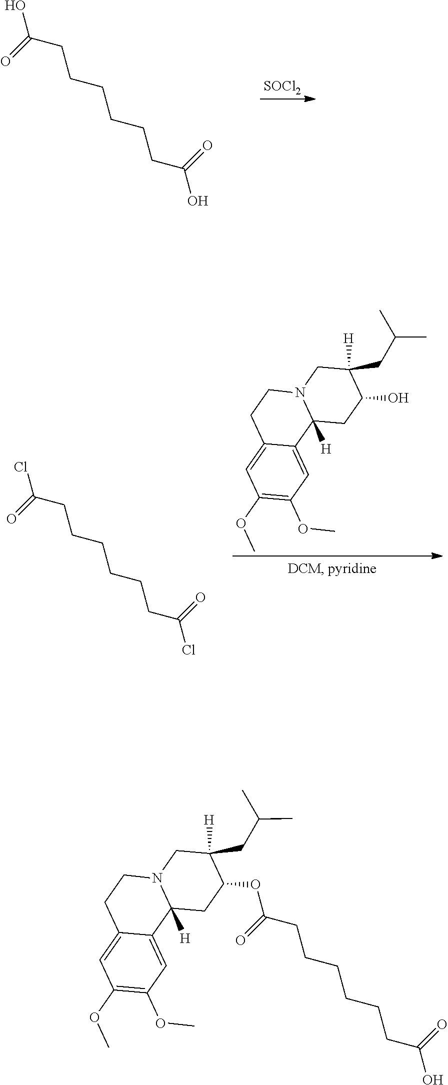 [SMILES: O=C(O)CCCCCCC(=O)O ; "SOCl2" ; O=C(Cl)CCCCCCC(=O)Cl ; COc1cc2c(cc1OC)[C@H]1C[C@@H](O)[C@H](CC(C)C)CN1CC2 ; COc1cc2c(cc1OC)[C@H]1C[C@@H](OC(=O)CCCCCCC(=O)O)[C@H](CC(C)C)CN1CC2]

### octanedioyl dichloride

A mixture of suberic acid (400 mg) in SOCl₂ (10 mL) was stirred at 90° C. for 4 h. The resulting mixture was concentrated under reduced pressure. This resulted in octanedioyl dichloride (462 mg, crude) as a colorless oil.

### 8-[[(2R,3R,11bR)-9,10-dimethoxy-3-(2-methylpropyl)-1H,2H,3H,4H,6H,7H,11bH-pyrido[2,1-a]isoquinolin-2-yl]oxy]-8-oxooctanoic acid

To a stirred mixture of (2R,3R,11bR)-9,10-dimethoxy-3-propyl-1H,2H,3H,4H,6H,7H,11bH-pyrido[2,1-a]isoquinolin-2-ol (400 mg, 1.310 mmol, 1 equiv) and pyridine (311 mg, 3.932 mmol, 3 equiv) in DCM (5 mL) was added octanedioyl dichloride (276 mg, 1.308 mmol, 1 equiv) at 0° C. The resulting mixture was stirred at room temperature for 14 h. The resulting mixture was concentrated under reduced pressure. The residue was purified by Prep-HPLC with MeCN/water (4/1) to afford 8-[[(2R,3R,11bR)-9,10-dimethoxy-3-(2-methylpropyl)-1H,2H,3H,4H,6H,7H,11bH-pyrido[2,1-a]isoquinolin-2-yl]oxy]-8-oxooctanoic acid (121.4 mg, 19.49%) as a white solid.

LCMS (ESI): [M+H]⁺: 476.3

¹H N 1H NMR (400 MHz, Methanol-d₄) δ 6.74 (s, 1H), 6.72 (s, 1H), 4.88-4.72 (m, 1H), 3.81-3.80 (m, 6H), 3.43-3.40 (m, 1H), 3.19-3.10 (m, 3H), 2.77-2.61 (m, 3H), 2.41-2.37 (m, 2H), 2.30-2.22 (m, 3H), 2.10-1.93 (m, 1H), 1.72-1.61 (m, 5H), 1.61-1.32 (m, 6H), 1.13-1.09 (m, 1H), 0.96-0.93 (m, 6H).

### Example 6: 5-([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)pyridine-3-carboxylic acid

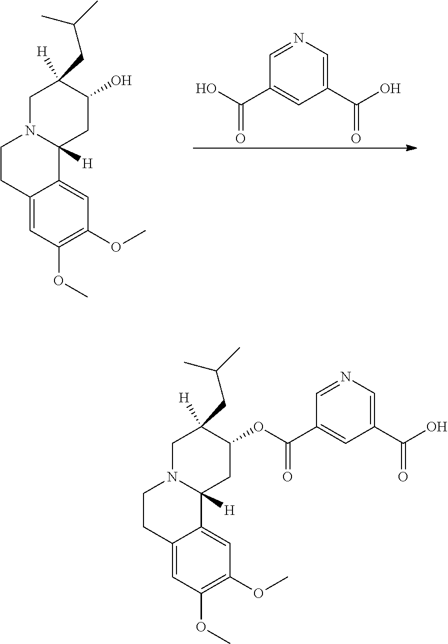 [SMILES: COc1cc2c(cc1OC)[C@H]1C[C@@H](O)[C@H](CC(C)C)CN1CC2 ; O=C(O)c1cncc(C(=O)O)c1 ; COc1cc2c(cc1OC)[C@H]1C[C@@H](OC(=O)c3cncc(C(=O)O)c3)[C@H](CC(C)C)CN1CC2]

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv), DMAP (191 mg, 1.563 mmol, 1 equiv) and 3,5-pyridinedicarboxylic acid (785 mg, 4.697 mmol, 3 equiv) in DCM (6 ml) was added EDCl (900 mg, 4.695 mmol, 3 equiv) at room temperature. The reaction was stirred at room temperature for 15 h. The resulting mixture was concentrated under vacuum. The residue was purified by Prep-HPLC with following conditions: Column: XBridge Prep OBD C18 Column, 19*250 mm, 5 um; Mobile Phase A: Water (10 MMOL/L NH₄HCO₃), Mobile Phase B: ACN; Flow rate: 25 mL/min; Gradient: 20 B to 50 B in 7 min; 254 nm. This resulted in 83.8 mg, (11.43%) 5-([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)pyridine-3-carboxylic acid as a light yellow solid.

LCMS (ESI): [M+H]⁺: 469.2

¹H NMR (300 MHz, Methanol-d₄) δ 9.29 (s, 1H), 9.21 (s, 1H), 8.91 (s, 1H), 6.81 (s, 1H), 6.79 (s, 1H), 5.18-5.17 (m, 1H), 4.10-3.97 (m, 1H), 3.82-3.80 (m, 6H), 3.56-3.48 (m, 2H), 3.22-3.10 (m, 1H), 3.08-2.90 (m, 2H), 2.88-2.73 (m, 1H), 2.34-2.31 (m, 1H), 1.98-1.71 (m, 2H), 1.52-1.42 (m, 1H), 1.42-1.04 (m, 2H), 1.04-0.94 (m, 6H).

### Example 7: (2R,3R,11bR)-9,10-dimethoxy-3-(2-methylpropyl)-1H,2H,3H,4H,6H,7H,11bH-pyrido[2,1-a]isoquinolin-2-yl 1-isopropyl butanedioate

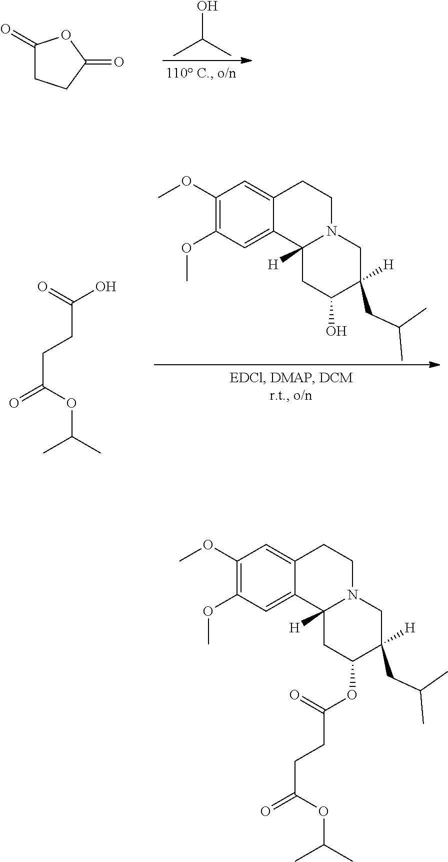

### 4-isopropoxy-4-oxobutanoic acid

A solution of succinic anhydride (1 g, 9.993 mmol, 1 equiv) and isopropyl alcohol (900 mg, 14.989 mmol, 1.5 equiv) was stirred at 110° C. overnight. The resulting mixture was concentrated under reduced pressure. This resulted in 1.2 g (crude) of 4-isopropoxy-4-oxobutanoic acid as a white oil.

### (2R,3R,11bR)-9,10-dimethoxy-3-(2-methylpropyl)-1H,2H,3H,4H,6H,7H,11bH-pyrido[2,1-a]isoquinolin-2-yl 1-isopropyl butanedioate

To a mixture of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) in DCM (10 mL) were added 4-isopropoxy-4-oxobutanoic acid (376 mg, 2.348 mmol, 1.5 equiv), EDCl (900 mg, 4.696 mmol, 3 equiv) and DMAP (191 mg, 1.565 mmol, 1 equiv). The resulting solution was stirred at room temperature overnight. The residue was applied onto a silica gel column with ethyl acetate/petroleum ether (2/1). The crude product (300 mg) was purified by Prep-HPLC with MeCN/H₂O=60/40. This resulted in 254.5 mg (34.87%) of (2R,3R,11bR)-9,10-dimethoxy-3-(2-methylpropyl)-1H,2H,3H,4H,6H,7H,11bH-pyrido[2,1-a]isoquinolin-2-yl 1-isopropyl butanedioate as a light yellow solid.

LCMS (ESI): [M+H]⁺: 462.4

¹H NMR (400 MHz, Methanol-d₄) δ 6.73 (s, 1H), 6.71 (s, 1H), 5.07-4.99 (m, 1H), 4.87-4.71 (m, 1H), 3.81 (s, 3H), 3.80 (s, 3H), 3.30-3.27 (m, 1H), 3.15-3.06 (m, 3H), 2.80-2.53 (m, 7H), 2.16 (t, J=11.7 Hz, 1H), 2.07-1.92 (m, 1H), 1.75-1.60 (m, 1H), 1.56-1.30 (m, 2H), 1.26 (d, J=6.2 Hz, 6H), 1.07-1.01 (m, 1H), 0.97-0.94 (m, 6H).

### Example 8: (2R,3R,11bR)-9,10-dimethoxy-3-(2-methylpropyl)-1H,2H,3H,4H,6H,7H,11bH-pyrido[2,1-a]isoquinolin-2-yl 1-isopropyl (2E)-but-2-enedioate

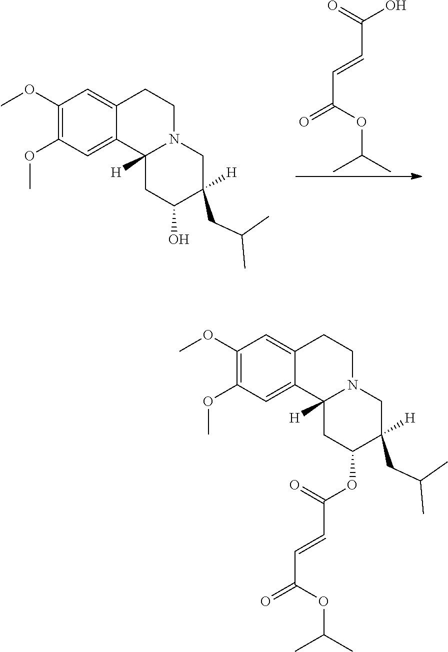

To a stirred mixture of (2R,3R,11bR)-9,10-dimethoxy-3-(2-methylpropyl)-1H,2H,3H,4H, 6H,7H,11bH-pyrido[2,1-a]isoquinolin-2-ol (500 mg, 1.565 mmol, 1 equiv), DMAP (191 mg, 1.563 mmol, 1 equiv) and (2E)-4-isopropoxy-4-oxobut-2-enoic acid (371 mg, 2.346 mmol, 1.5 equiv) in DCM (5 mL) was added EDCl (900 mg, 4.695 mmol, 3 equiv) at room temperature. The resulting mixture was stirred at room temperature for 14 h. The reaction was concentrated. The residue was purified by silica gel column chromatography, eluted with PE/EtOAc (3/1) to afford (2R,3R,11bR)-9,10-dimethoxy-3-(2-methylpropyl)-1H,2H,3H,4H,6H,7H,11bH-pyrido[2,1-a]isoquinolin-2-yl 1-isopropyl (2E)-but-2-enedioate (259.4 mg, 35.70%) as a light yellow solid.

LCMS (ESI): [M+H]⁺: 460.3

¹H NMR (300 MHz, Methanol-d₄) δ 6.83 (s, 2H), 6.73 (s, 1H), 6.70 (s, 1H), 5.14-5.06 (m, 1H), 4.86-4.80 (m, 2H), 3.80-3.78 (m, 6H), 3.33-3.04 (m, 3H), 2.75-2.70 (m, 2H), 2.70-2.53 (m, 1H), 2.22-2.18 (m, 1H), 2.18-2.08 (m, 1H), 1.78-1.60 (m, 1H), 1.60-1.52 (m, 1H), 1.35-1.26 (m, 7H), 1.15-1.10 (m, 1H), 0.95-0.91 (m, 6H).

### Example 9: 3-((2R,3R,11bR)-3-isobutyl-9,10-dimethoxy-1,3,4,6,7,11b-hexahydro-2H-pyrido[2,1-a]isoquinolin-2-yl) 5-isopropyl pyridine-3,5-dicarboxylate

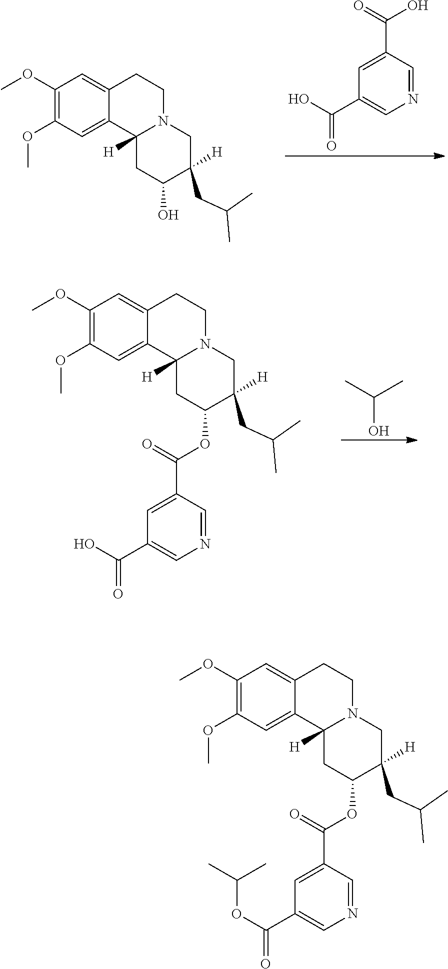

### 5-((((2R,3R,11bR)-3-isobutyl-9,10-dimethoxy-1,3,4,6,7,11b-hexahydro-2H-pyrido[2,1-a]isoquinolin-2-yl)oxy)carbonyl)nicotinic acid

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv), DMAP (191 mg, 1.563 mmol, 1 equiv) and 3,5-pyridinedicarboxylic acid (785 mg, 4.697 mmol, 3 equiv) in DCM (5 mL) was added EDCl (900 mg, 4.695 mmol, 3 equiv) at room temperature. The reaction was stirred at room temperature for 15 h. The residue was purified by silica gel column chromatography, eluted with DCM/MeOH (4/1) to afford 5-((((2R,3R,11bR)-3-isobutyl-9,10-dimethoxy-1,3,4,6,7,11b-hexahydro-2H-pyrido[2,1-a]isoquinolin-2-yl)oxy)carbonyl)nicotinic acid (195 mg, 26.32%) as a light yellow solid.

### 3-((2R,3R,11bR)-3-isobutyl-9,10-dimethoxy-1,3,4,6,7,11b-hexahydro-2H-pyrido[2,1-a]isoquinolin-2-yl) 5-isopropyl pyridine-3,5-dicarboxylate

To a stirred mixture of 5-([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)pyridine-3-carboxylic acid (195 mg, 0.416 mmol, 1 equiv) and isopropyl alcohol (250 mg, 4.160 mmol, 10 equiv) in DCM (3 mL) was added DMAP (51 mg, 0.417 mmol, 1 equiv) and EDCl (239 mg, 1.247 mmol, 3 equiv) at room temperature. The reaction was stirred at room temperature for 15 h. The resulting mixture was concentrated under vacuum. The residue was purified by Prep-HPLC with MeCN/water (7/3). This resulted in 3-((2R,3R,11bR)-3-isobutyl-9,10-dimethoxy-1,3,4,6,7,11b-hexahydro-2H-pyrido[2,1-a]isoquinolin-2-yl) 5-isopropyl pyridine-3,5-dicarboxylate (60.8 mg, 27.74%) as a white solid.

LCMS (ESI): [M+H]⁺: 511.4

¹H NMR (400 MHz, Methanol-d₄) δ 9.34-9.32 (m, 2H), 8.88 (s, 1H), 6.77 (s, 1H), 6.72 (s, 1H), 5.33-5.30 (m, 1H), 5.03-5.02 (m, 1H), 3.81-3.79 (m, 6H), 3.42-3.32 (m, 2H), 3.20-3.18 (m, 1H), 3.15-3.11 (m, 1H), 2.91-2.85 (m, 1H), 2.85-2.77 (m, 1H), 2.73-2.58 (m, 1H), 2.27-2.25 (m, 2H), 1.81-1.66 (m, 2H), 1.44-1.39 (m, 7H), 1.21-1.19 (m, 1H), 1.01-0.95 (m, 6H).

### Example 10: (2R,3R,11bR)-9,10-dimethoxy-3-(2-methylpropyl)-1H,2H,3H,4H,6H,7H,11bH-pyrido[2,1-a]isoquinolin-2-yl 1-isopropyl (2Z)-but-2-enedioate

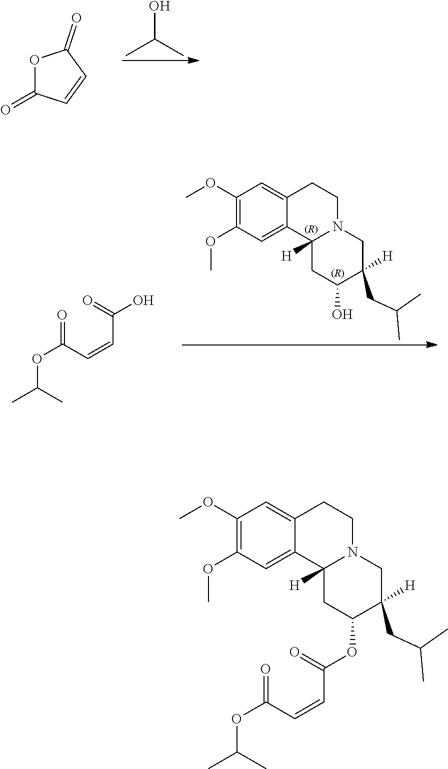

### (2Z)-4-isopropoxy-4-oxobut-2-enoic acid

IPA (0.76 g, 0.010 mmol, 1 equiv) was added to maleic anhydride (1.00 g, 0.010 mmol, 1 equiv) at 70° C. with stirring. The resulting mixture was stirred at room temperature for 15 h. The resulting mixture was concentrated under reduced pressure. This resulted in (1.595 g, crude) of (2Z)-4-isopropoxy-4-oxobut-2-enoic acid as a colorless oil.

### (2R,3R,11bR)-9,10-dimethoxy-3-(2-methylpropyl)-1H,2H,3H,4H,6H,7H,11bH-pyrido[2,1-a]isoquinolin-2-yl 1-isopropyl (2Z)-but-2-enedioate

To a stirred mixture of (2R,3R,11bR)-9,10-dimethoxy-3-(2-methylpropyl)-1H,2H,3H,4H,6H, 7H,11bH-pyrido[2,1-a]isoquinolin-2-ol (500 mg, 1.565 mmol, 1 equiv), DMAP (191 mg, 1.563 mmol, 1 equiv) and (2Z)-4-isopropoxy-4-oxobut-2-enoic acid (371 mg, 2.346 mmol, 1.5 equiv) in DCM (10 mL) was added EDCl (900 mg, 4.695 mmol, 3 equiv) at room temperature. The resulting mixture was stirred at room temperature for 14 h. The resulting mixture was concentrated under reduced pressure. The residue was purified by Prep-HPLC with MeCN/water (3/1) to afford (2R,3R,11bR)-9,10-dimethoxy-3-(2-methylpropyl)-1H,2H,3H,4H,6H,7H,11bH-pyrido[2,1-a]isoquinolin-2-yl 1-isopropyl (2Z)-but-2-enedioate (217.7 mg, 30.11%) as a light yellow solid.

LCMS (ESI): [M+H]⁺: 460.4

¹H NMR (300 MHz, Methanol-d₄) δ 6.83 (s, 2H), 6.74 (s, 1H), 6.71 (s, 1H), 5.12-5.08 (m, 1H), 4.86-4.82 (m, 2H), 3.80 (s, 3H), 3.78 (s, 3H), 3.16-3.04 (m, 3H), 2.75-2.72 (m, 2H), 2.72-2.53 (m, 1H), 2.23-2.19 (m, 1H), 2.19-2.08 (m, 1H), 1.80-1.62 (m, 1H), 1.62-1.57 (m, 1H), 1.35-1.26 (m, 7H), 1.15-1.10 (m, 1H), 0.95-0.91 (m, 6H).

### Example 11: (2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid

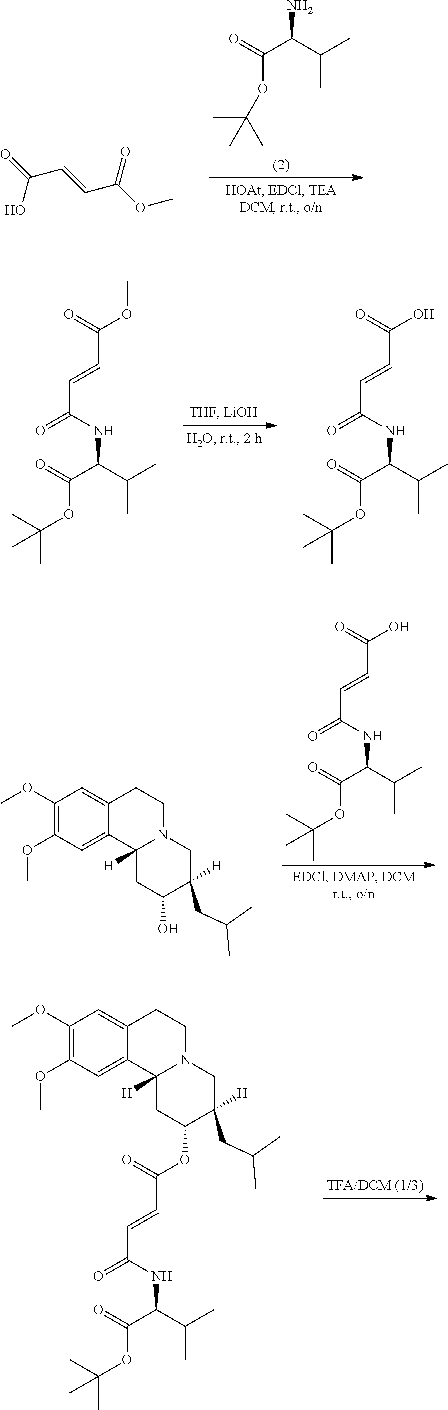

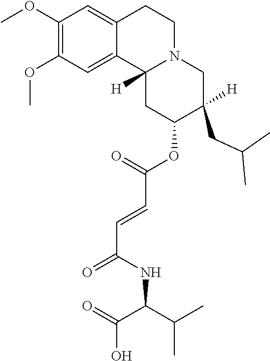 [SMILES: COc1cc2c(cc1OC)[C@H]1C[C@@H](OC(=O)/C=C/C(=O)N[C@H](C(=O)O)C(C)C)[C@H](CC(C)C)CN1CC2]

### Methyl (2E)-3-[[(2S)-1-(tert-butoxy)-3-methyl-1-oxobutan-2-yl]carbamoyl]prop-2-enoate

To a solution of (2E)-4-methoxy-4-oxobut-2-enoic acid (3 g, 23.059 mmol, 1 equiv) in DCM (30 mL) were added HOAt (3.1 g, 23.059 mmol, 1 equiv), EDCl (4.4 g, 23.059 mmol, 1 equiv), tert-butyl (2S)-2-amino-3-methylbutanoate (2.8 g, 16.161 mmol, 0.7 equiv) and TEA (2.6 g, 25.694 mmol, 1.1 equiv) at 0° C. The resulting solution was stirred at room temperature overnight. The reaction was concentrated. The residue was applied onto a silica gel column with ethyl acetate/petroleum ether (1/3). This resulted in 4.3 g of methyl (2E)-3-[[(2S)-1-(tert-butoxy)-3-methyl-1-oxobutan-2-yl]carbamoyl]prop-2-enoate as a white solid.

### (2E)-3-[[(2S)-1-(tert-butoxy)-3-methyl-1-oxobutan-2-yl]carbamoyl]prop-2-enoic acid

To a solution of methyl (2E)-3-[[(2S)-1-(tert-butoxy)-3-methyl-1-oxobutan-2-yl]carbamoyl]prop-2-enoate (1 g, 3.505 mmol, 1 equiv) in THF (15 mL) was added LiH (567 mg, 23.656 mmol, 6.7 equiv) and H₂O (5 mL). The resulting solution was stirred at room temperature for 2 h. The pH value of the solution was adjusted to 5 with HCl (1 mol/L). The resulting mixture was extracted with 3×15 mL of ethyl acetate. The combined organic layers were dried over anhydrous sodium sulfate, filtered and concentrated under vacuum. This resulted in 976 mg (97.51%) of (2E)-3-[[(2S)-1-(tert-butoxy)-3-methyl-1-oxobutan-2-yl]carbamoyl]prop-2-enoic acid as a white solid.

### (2R,3R,11bR)-9,10-dimethoxy-3-(2-methylpropyl)-1H,2H,3H,4H,6H,7H,11bH-pyrido[2,1-a]isoquinolin-2-yl (2E)-3-[[(2S)-1-isopropoxy-3-methyl-1-oxobutan-2-yl]carbamoyl]prop-2-enoate

To a stirred solution of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) in DCM (20 mL) was added EDCl (900 mg, 4.696 mmol, 3 equiv), DMAP (191 mg, 1.565 mmol, 1 equiv) and (2E)-3-[[(2S)-1-(tert-butoxy)-3-methyl-1-oxobutan-2-yl]carbamoyl]prop-2-enoic acid (637 mg, 2.348 mmol, 1.5 equiv). The resulting mixture was stirred at room temperature overnight. The residue was purified by silica gel column chromatography, eluted with ethyl acetate/petroleum ether (1/2). This resulted in 295 mg of (2R,3R,11bR)-9,10-dimethoxy-3-(2-methylpropyl)-1H,2H,3H,4H,6H,7H,11bH-pyrido[2,1-a]isoquinolin-2-yl (2E)-3-[[(2S)-1-isopropoxy-3-methyl-1-oxobutan-2-yl]carbamoyl]prop-2-enoate as a light yellow solid.

LCMS (ESI): [M+H]⁺: 573.5

### (2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoicacid

To a stirred solution of (2R,3R,11bR)-9,10-dimethoxy-3-(2-methylpropyl)-1H,2H,3H,4H,6H,7H,11bH-pyrido[2,1-a]isoquinolin-2-yl (2E)-3-[[(2S)-1-(tert-butoxy)-3-methyl-1-oxobutan-2-yl]carbamoyl]prop-2-enoate (295 mg, 0.515 mmol, 1 equiv) in DCM (3 mL) was added TFA (1 mL). The resulting solution was stirred at room temperature overnight. The resulting mixture was concentrated. The crude product was purified by Flash-Prep-HPLC with MeCN/H₂O=30/70. This resulted in 136.6 mg (51.33%) of (2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid as a light yellow solid.

LCMS (ESI): [M+H]⁺: 517.5

¹H NMR (400 MHz, Methanol-d₄) δ 7.28 (d, J=15.5 Hz, 1H), 6.79-6.75 (m, 3H), 4.96-4.87 (m, 1H), 4.41 (d, J=5.3 Hz, 1H), 3.87-3.82 (m, 7H), 3.35-3.32 (m, 2H), 3.18-3.15 (m, 1H), 2.92-2.84 (m, 3H), 2.63-2.57 (m, 1H), 2.34-2.05 (m, 2H), 1.73-1.67 (m, 2H), 1.51-1.36 (m, 1H), 1.17-1.13 (m, 1H), 1.00-0.94 (m, 12H).

### Example 12: (2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid

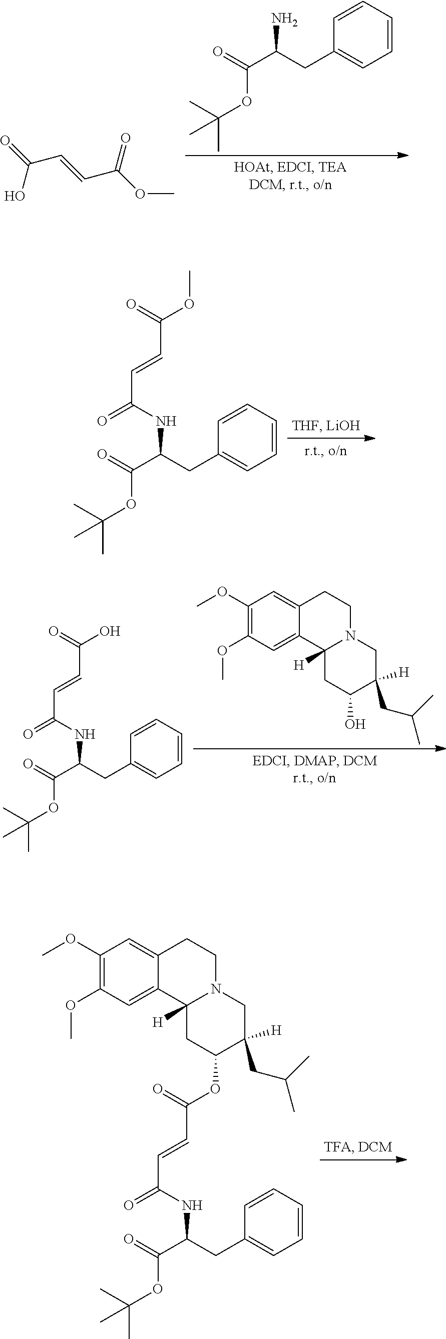

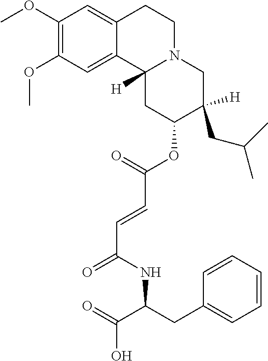 [SMILES: COc1cc2c(cc1OC)[C@H]1C[C@@H](OC(=O)/C=C/C(=O)N[C@@H](Cc3ccccc3)C(=O)O)[C@H](CC(C)C)CN1CC2]

### methyl (2E)-3-[[(2S)-1-(tert-butoxy)-1-oxo-3-phenylpropan-2-yl]carbamoyl]prop-2-enoate

To a stirred mixture of (2E)-4-methoxy-4-oxobut-2-enoic acid (3 g, 23.059 mmol, 1 equiv), HOAt (3.14 g, 23.062 mmol, 1 equiv) and tert-butyl (2S)-2-amino-3-phenylpropanoate (3.57 g, 16.141 mmol, 0.7 equiv) in DCM (20 mL) was added TEA (2.57 g, 25.358 mmol, 1.1 equiv) and EDCl (4.42 g, 23.057 mmol, 1 equiv) at room temperature. The resulting mixture was stirred at room temperature for 15 h. The resulting mixture was concentrated under reduced pressure. The residue was purified by silica gel column chromatography, eluted with PE/EtOAc (3/1) to afford methyl (2E)-3-[[(2S)-1-(tert-butoxy)-1-oxo-3-phenylpropan-2-yl]carbamoyl]prop-2-enoate (3.39 g, 44.23%) as a white solid.

### (2E)-3-[[(2S)-1-(tert-butoxy)-1-oxo-3-phenylpropan-2-yl]carbamoyl]prop-2-enoic acid

To a stirred mixture of methyl (2E)-3-[[(2S)-1-(tert-butoxy)-1-oxo-3-phenylpropan-2-yl]carbamoyl]prop-2-enoate (1 g, 3.000 mmol, 1 equiv) in THF (60 mL) was added a solution of LiH (520 mg, 21.714 mmol, 7.24 equiv) in H₂O (20 mL) at room temperature. The resulting mixture was stirred at room temperature for 15 h. The mixture was acidified to pH 4 with HCl. The precipitated solids were collected by filtration. This resulted in (750 mg, 78.29%) of (2E)-3-[[(2S)-1-(tert-butoxy)-1-oxo-3-phenylpropan-2-yl]carbamoyl]prop-2-enoic acid as a white solid.

### (2R,3R,11bR)-9,10-dimethoxy-3-(2-methylpropyl)-1H,2H,3H,4H,6H,7H,11bH-pyrido[2,1-a]isoquinolin-2-yl (2E)-3-[[(2S)-1-(tert-butoxy)-1-oxo-3-phenylpropan-2-yl]carbamoyl]prop-2-enoate

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) and (2E)-3-[[(2S)-1-(tert-butoxy)-1-oxo-3-phenylpropan-2-yl]carbamoyl]prop-2-enoic acid (750 mg, 2.348 mmol, 1.5 equiv) in DCM (9 mL) was added DMAP (192 mg, 1.572 mmol, 1 equiv) and EDCl (900 mg, 4.695 mmol, 3 equiv) at room temperature. The resulting mixture was stirred at room temperature for 15 h. The resulting mixture was concentrated under reduced pressure. The residue was purified by silica gel column with EA/PE (1/1). This resulted in (240 mg, 24.70%) of (2R,3R,11bR)-9,10-dimethoxy-3-(2-methylpropyl)-1H,2H,3H,4H,6H,7H,11bH-pyrido[2,1-a]isoquinolin-2-yl (2E)-3-[[(2S)-1-(tert-butoxy)-1-oxo-3-phenylpropan-2-yl]carbamoyl]prop-2-enoate as a light yellow solid.

### (2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid

To a stirred mixture of (2R,3R,11bR)-9,10-dimethoxy-3-(2-methylpropyl)-1H,2H,3H,4H,6H,7H,11bH-pyrido[2,1-a]isoquinolin-2-yl (2E)-3-[[(2S)-1-(tert-butoxy)-1-oxo-3-phenylpropan-2-yl]carbamoyl]prop-2-enoate (240 mg, 0.387 mmol, 1 equiv) in DCM (1 mL) was added TFA (1 mL) at room temperature. The reaction was stirred at room temperature for 12 h. The resulting mixture was concentrated under reduced pressure. The residue was purified by Prep-HPLC with following conditions: Column: XBridge Prep OBD C18 Column, 30×150 mm 5 um; Mobile Phase A: Water (10 MMOL/L NH₄HCO₃), Mobile Phase B: ACN; Flow rate:60 mL/min; Gradient:25 B to 48 B in 7 min; 254/220 nm. This resulted in (2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid (69.3 mg, 31.70%) as a light yellow solid.

LCMS (ESI): [M+H]⁺: 565.3

¹H NMR (400 MHz, Methanol-d₄) δ 7.25-7.17 (m, 5H), 7.07 (d, J=15.2 Hz, 1H), 6.75 (s, 2H), 6.67 (d, J=15.6 Hz, 1H), 4.91-4.85 (m, 1H), 4.68-4.65 (m, 1H), 3.84-3.79 (m, 1H), 3.79-3.78 (m, 6H), 3.40-3.31 (m, 2H), 3.30-3.28 (m, 1H), 3.27-3.16 (m, 1H), 3.00-2.84 (m, 4H), 2.62-2.57 (m, 1H), 2.21-2.10 (m, 1H), 1.70-1.64 (m, 2H), 1.41-1.30 (m, 1H), 1.25-1.12 (m, 1H), 0.94-0.90 (m, 6H).

### Example 13: (2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid

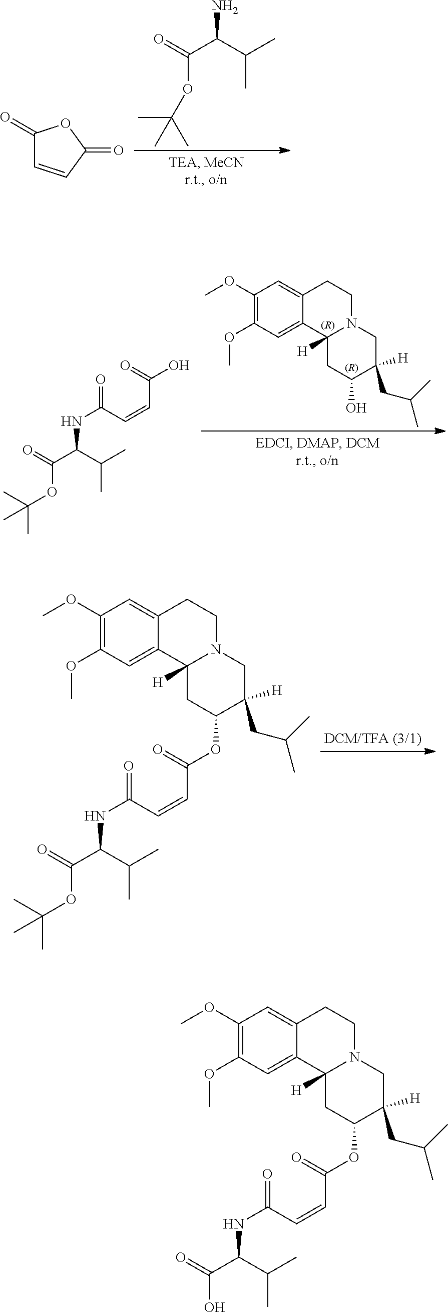

### (2Z)-3-[[(2S)-1-(tert-butoxy)-3-methyl-1-oxobutan-2-yl]carbamoyl]prop-2-enoic acid

To a stirred mixture of maleic anhydride (0.56 g, 5.711 mmol, 1 equiv) in MeCN (6 mL) was added TEA (0.58 g, 5.771 mmol, 1 equiv) and tert-butyl (2S)-2-amino-3-methylbutanoate (1.00 g, 5.772 mmol, 1 equiv) at room temperature. The resulting mixture was stirred at room temperature for 14 h. The resulting mixture was concentrated under reduced pressure. The resulting mixture was dissolved in ethyl acetate and washed with aqueous KHSO₄ (5%). The organic layers were dried over Na₂SO₄, filtered and concentrated under vacuum. This resulted in (2Z)-3-[[(2S)-1-(tert-butoxy)-3-methyl-1-oxobutan-2-yl]carbamoyl]prop-2-enoic acid (1.229 g, 79.32%) as a white solid.

### (2R,3R,11bR)-9,10-dimethoxy-3-(2-methylpropyl)-1H,2H,3H,4H,6H,7H,11bH-pyrido[2,1-a]isoquinolin-2-yl(2Z)-3-[[(2S)-1-(tert-butoxy)-3-methyl-1-oxobutan-2-yl]carbamoyl]prop-2-enoate

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (400 mg, 1.252 mmol, 1 equiv), DMAP (153 mg, 1.252 mmol, 1 equiv) and (2Z)-3-[[(2S)-1-(tert-butoxy)-3-methyl-1-oxobutan-2-yl]carbamoyl]prop-2-enoic acid (510 mg, 1.880 mmol, 1.5 equiv) in DCM (5 mL) was added EDCl (720 mg, 3.756 mmol, 3 equiv) at room temperature. The resulting mixture was stirred at room temperature for 14 h. The resulting mixture was concentrated under vacuum. The residue was purified by silica gel column chromatography, eluted with PE/EA (2/1) to afford (2R,3R,11bR)-9,10-dimethoxy-3-(2-methylpropyl)-1H,2H,3H,4H,6H,7H,11bH-pyrido[2,1-a]isoquinolin-2-yl (2Z)-3-[[(2S)-1-(tert-butoxy)-3-methyl-1-oxobutan-2-yl]carbamoyl]prop-2-enoate (223 mg, 31.09%) as a light yellow solid.

### (2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoicacid

To a stirred mixture of (2R,3R,11bR)-9,10-dimethoxy-3-(2-methylpropyl)-1H,2H,3H,4H,6H,7H,11bH-pyrido[2,1-a]isoquinolin-2-yl (2Z)-3-[[(2S)-1-(tert-butoxy)-3-methyl-1-oxobutan-2-yl]carbamoyl]prop-2-enoate (223 mg, 1 equiv) in DCM (3 mL) was added TFA (1 mL) dropwise at 0° C. The resulting mixture was stirred at room temperature for 14 h. The resulting mixture was concentrated under reduced pressure. The residue was purified by Prep-HPLC with following conditions: Column: Xselect CSH OBD Column 30*150 mm 5 um, n; Mobile Phase A: Water (0.1% FA), Mobile Phase B: ACN; Flow rate:60 mL/min; Gradient:12 B to 54 B in 7 min; 254 nm. This resulted in (2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid (62.8 mg, 29.97%) as a light yellow solid.

LCMS (ESI): [M+H]⁺: 517.3

¹H NMR (400 MHz, Methanol-d₄) δ 7.28 (d, J=15.2 Hz, 1H), 6.80-6.76 (m, 3H), 4.95-4.92 (m, 1H), 4.42-4.41 (m, 1H), 3.82-3.81 (m, 7H), 3.39-3.36 (m, 2H), 3.17-3.14 (m, 1H), 2.88-2.84 (m, 3H), 2.64-2.57 (m, 1H), 2.27-2.13 (m, 2H), 1.74-1.68 (m, 2H), 1.38-1.36 (m, 1H), 1.19-1.16 (m, 1H), 1.01-0.94 (m, 12H).

### Example 14: (2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid

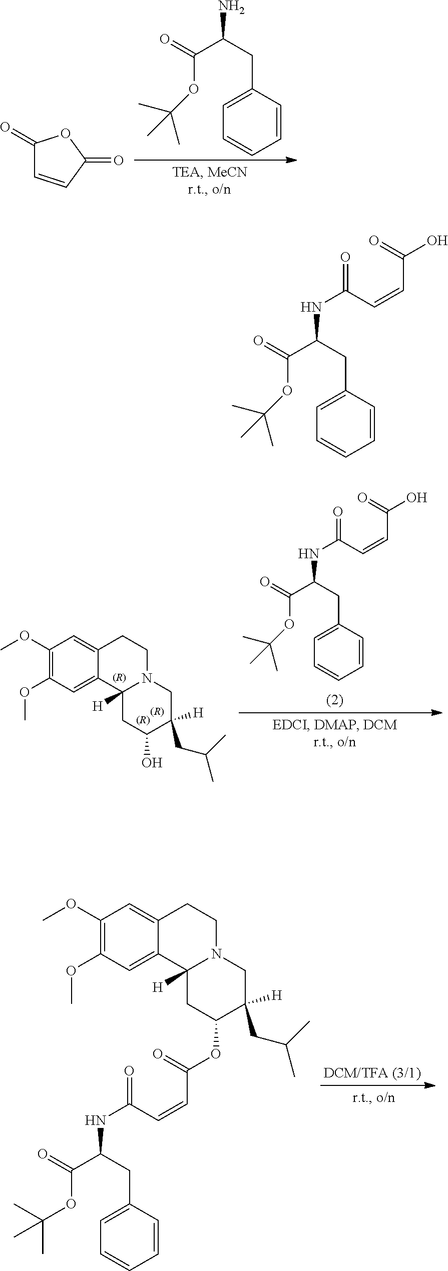

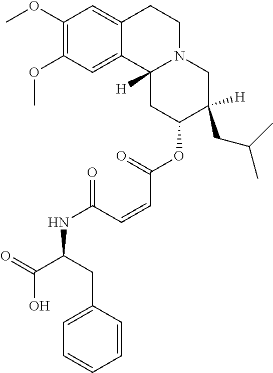 [SMILES: COc1cc2c(cc1OC)[C@H]1C[C@@H](OC(=O)/C=C\C(=O)N[C@@H](Cc3ccccc3)C(=O)O)[C@H](CC(C)C)CN1CC2]

### (S,Z)-4-((1-(tert-butoxy)-1-oxo-3-phenylpropan-2-yl)amino)-4-oxobut-2-enoic acid

To a stirred solution of maleic anhydride (443 mg, 4.518 mmol, 1 equiv) and tert-butyl (2S)-2-amino-3-phenylpropanoate (1 g, 4.519 mmol, 1 equiv) in MeCN (8 mL) was added TEA (457 mg, 4.516 mmol, 1 equiv). The resulting mixture was stirred at room temperature overnight. The resulting mixture was dissolved in ethyl acetate and washed with aqueous KHSO₄ (5%). The organic layers were dried over Na₂SO₄, filtered and concentrated under vacuum. This resulted in 893 mg (crude) of (S,Z)-4-((1-(tert-butoxy)-1-oxo-3-phenylpropan-2-yl)amino)-4-oxobut-2-enoic acid as a white solid.

### (2R,3R,11bR)-9,10-dimethoxy-3-(2-methylpropyl)-1H,2H,3H,4H,6H,7H,11bH-pyrido[2,1-a]isoquinolin-2-yl(2Z)-3-[[(2S)-1-(tert-butoxy)-1-oxo-3-phenylpropan-2-yl]carbamoyl]prop-2-enoate

To a mixture of (2R,3R,11bR)-9,10-dimethoxy-3-(2-methylpropyl)-1H,2H,3H,4H,6H,7H,11bH-pyrido[2,1-a]isoquinolin-2-ol (400 mg, 1.252 mmol, 1 equiv) and (2Z)-3-[[(2S)-1-(tert-butoxy)-1-oxo-3-phenylpropan-2-yl]carbamoyl]prop-2-enoic acid (600 mg, 1.878 mmol, 1.5 equiv) in DCM (5.5 mL) were added EDCl (720 mg, 3.757 mmol, 3 equiv) and DMAP (153 mg, 1.252 mmol, 1 equiv). The resulting mixture was stirred at room temperature overnight. The resulting mixture was concentrated under vacuum. The residue was applied onto a silica gel column with ethyl acetate/petroleum ether (1/2). This resulted in 260 mg (33.45%) (2R,3R,11bR)-9,10-dimethoxy-3-(2-methylpropyl)-1H,2H,3H,4H,6H,7H,11bH-pyrido[2,1-a]isoquinolin-2-yl (2Z)-3-[[(2S)-1-(tert-butoxy)-1-oxo-3-phenylpropan-2-yl]carbamoyl]prop-2-enoate as a purple solid.

LCMS (ESI): [M+H]⁺: 621.5

### (2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid

To a stirred solution of (2R,3R,11bR)-9,10-dimethoxy-3-(2-methylpropyl)-1H,2H,3H,4H,6H,7H,11bH-pyrido[2,1-a]isoquinolin-2-yl (2Z)-3-[[(2S)-1-(tert-butoxy)-1-oxo-3-phenylpropan-2-yl]carbamoyl]prop-2-enoate (260 mg, 0.419 mmol, 1 equiv) in DCM (3 mL) was added TFA (1 mL). The resulting solution was stirred at room temperature overnight. The resulting mixture was concentrated. The crude product was purified by Prep-HPLC with MeCN/H₂O=25/75. This resulted in 123.4 mg (52.18%) of (2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid as a light yellow solid.

LCMS (ESI): [M+H]⁺: 565.5

¹H NMR (400 MHz, Methanol-d₄) δ 7.27-7.19 (m, 5H), 7.10 (d, J=15.5 Hz, 1H), 6.78 (d, J=2.6 Hz, 2H), 6.68 (d, J=15.5 Hz, 1H), 4.95-4.88 (m, 1H), 4.88-4.67 (m, 1H), 3.98-3.91 (m, 1H), 3.82-3.81 (m, 6H), 3.47-3.40 (m, 2H), 3.30-3.12 (m, 2H), 3.02-2.85 (m, 4H), 2.70-2.60 (m, 1H), 2.29-2.10 (m, 1H), 1.75-1.69 (m, 2H), 1.37-1.30 (m, 1H), 1.20-1.15 (m, 1H), 0.96-0.93 (m, 6H).

### Example 15: 3,5-bis[(2R,3R,11bR)-9,10-dimethoxy-3-(2-methylpropyl)-1H,2H,3H,4H,6H,7H,11bH-pyrido[2,1-a]isoquinolin-2-yl] pyridine-3,5-dicarboxylate

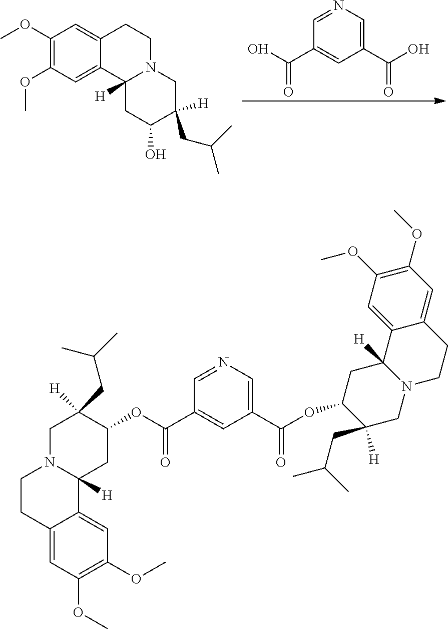 [SMILES: COc1cc2c(cc1OC)[C@H]1C[C@@H](O)[C@H](CC(C)C)CN1CC2 ; O=C(O)c1cncc(C(=O)O)c1 ; COc1cc2c(cc1OC)[C@H]1C[C@@H](OC(=O)c3cncc(C(=O)O[C@@H]4C[C@@H]5c6cc(OC)c(OC)cc6CCN5C[C@H]4CC(C)C)c3)[C@H](CC(C)C)CN1CC2]

To a stirred solution of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) in DCM (10 mL) was added EDCl (900 mg, 4.696 mmol, 3 equiv), DMAP (382.4 mg, 3.130 mmol, 2 equiv) and 3,5-pyridinedicarboxylic acid (157 mg, 0.939 mmol, 0.6 equiv). The resulting solution was stirred at room temperature overnight. The residue was applied onto a silica gel column with ethyl acetate/petroleum ether (1/1). The crude was purified by Prep-HPLC with MeCN/H₂O=70/30 to afford 3,5-bis[(2R,3R,11bR)-9,10-dimethoxy-3-(2-methylpropyl)-1H,2H,3H,4H,6H,7H,11bH-pyrido[2,1-a]isoquinolin-2-yl] pyridine-3,5-dicarboxylate 102.8 mg (14.2%) as a white solid.

LCMS (ESI): [M+H]⁺: 770.6

¹H NMR (400 MHz, Methanol-d₄) δ 9.35 (s, 1H), 9.34 (s, 1H), 8.90 (s, 1H), 6.77 (s, 2H), 6.72 (s, 2H), 4.92-5.02 (m, 2H), 3.78-3.76 (m, 12H), 3.39-3.30 (m, 2H), 3.23-3.04 (m, 6H), 2.92-2.81 (m, 2H), 2.80-2.68 (m, 2H), 2.66-2.49 (m, 2H), 2.27-2.11 (m, 4H), 1.83-1.58 (m, 4H), 1.39-1.33 (m, 2H), 1.21-1.16 (m, 2H), 0.95 (d, J=6.6 Hz, 12H).

### Example 16: bis[(2R,3R,11bR)-9,10-dimethoxy-3-(2-methylpropyl)-1H,2H,3H,4H,6H,7H,11bH-pyrido[2,1-a]isoquinolin-2-yl] octanedioate

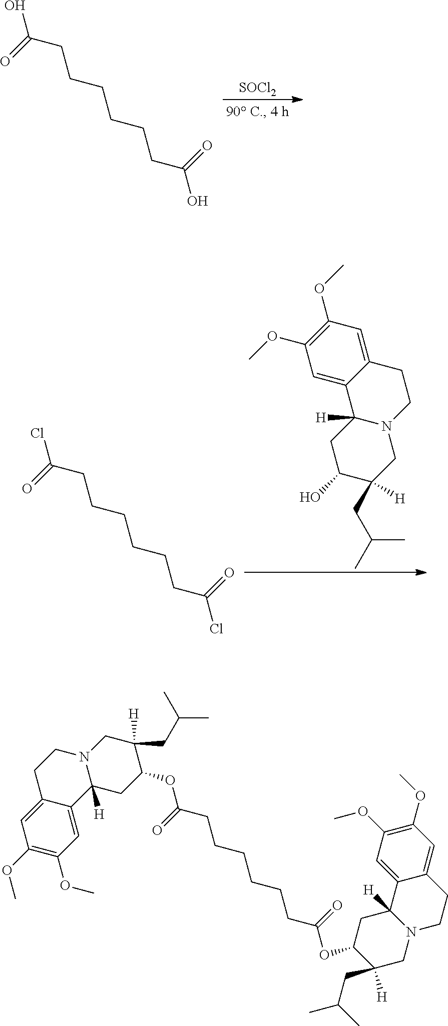 [SMILES: O=C(O)CCCCCCC(=O)O ; "SOCl2" ; O=C(Cl)CCCCCCC(=O)Cl ; COc1cc2c(cc1OC)[C@H]1C[C@@H](O)[C@H](CC(C)C)CN1CC2 ; COc1cc2c(cc1OC)[C@H]1C[C@@H](OC(=O)CCCCCCC(=O)O[C@@H]3C[C@@H]4c5cc(OC)c(OC)cc5CCN4C[C@H]3CC(C)C)[C@H](CC(C)C)CN1CC2]

### octanedioyl dichloride

A mixture of suberic acid (350 mg) in SOCl₂ (10 mL) was stirred at 90° C. for 4 h. The resulting mixture was concentrated under reduced pressure. This resulted in octanedioyl dichloride (363 mg, crude) as a colorless oil.

### bis[(2R,3R,11bR)-9,10-dimethoxy-3-(2-methylpropyl)-1H,2H,3H,4H,6H,7H,11bH-pyrido[2,1-a]isoquinolin-2-yl]octanedioate

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (550 mg, 1.722 mmol, 1 equiv) and pyridine (409 mg, 5.171 mmol, 3 equiv) in DCM (6 ml) was added octanedioyl dichloride (363 mg, 1.720 mmol, 1 equiv) at 0° C. The reaction was stirred at room temperature for 2 h. The resulting mixture was concentrated under vacuum. The residue was purified by Prep-HPLC with following conditions: Column: XBridge Shield RP18 OBD Column, 19*250 mm, 10 um; Mobile Phase A: Water (10 MMOL/L NH₄HCO₃), Mobile Phase B: ACN; Flow rate:25 mL/min; Gradient:40 B to 95 B in 7 min; 254/220 nm. This resulted in bis[(2R,3R,11bR)-9,10-dimethoxy-3-(2-methylpropyl)-1H,2H,3H,4H,6H,7H,11bH-pyrido[2,1-a]isoquinolin-2-yl] octanedioate (183.7 mg, 13.73%) as a light yellow solid.

LCMS (ESI): [M+H]⁺: 777.6

¹H NMR (300 MHz, Methanol-d₄) δ 6.66 (s, 2H), 6.64 (s, 2H), 4.67-4.66 (m, 2H), 3.78-3.75 (m, 12H), 3.31-2.92 (m, 8H), 2.79-2.59 (m, 4H), 2.59-2.48 (m, 2H), 2.39-2.34 (m, 4H), 2.15-2.07 (m, 2H), 2.02-1.85 (m, 2H), 1.71-1.62 (m, 6H), 1.47-1.32 (m, 6H), 1.28-1.24 (m, 2H), 1.10-1.05 (m, 2H), 0.93-0.89 (m, 12H).

### Example 17: [(2R,3R,11bR)-9,10-dimethoxy-3-(2-methylpropyl)-1H,2H,3H,4H,6H,7H,11bH-pyrido[2,1-a]isoquinolin-2-yl]butanedioate

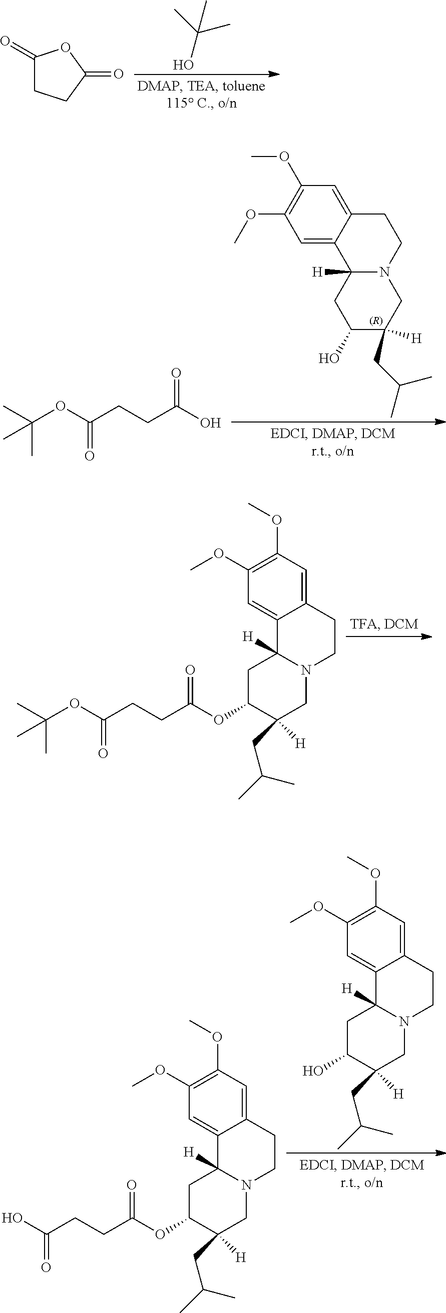

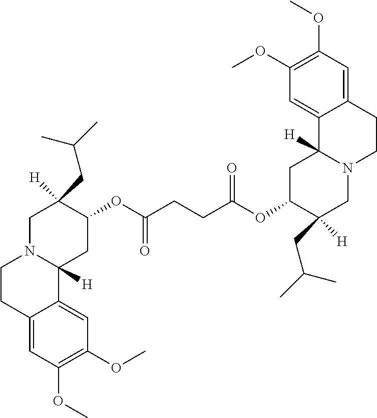 [SMILES: COc1cc2c(cc1OC)[C@H]1C[C@@H](OC(=O)CCC(=O)O[C@@H]3C[C@@H]4c5cc(OC)c(OC)cc5CCN4C[C@H]3CC(C)C)[C@H](CC(C)C)CN1CC2]

### 4-(tert-butoxy)-4-oxobutanoic acid

To a stirred mixture of succinic anhydride (3 g, 29.978 mmol, 1 equiv), TEA (910 mg, 8.993 mmol, 0.3 equiv) and 2-methyl-2-propanol (35 mL, 89.934 mmol, 3 equiv) in toluene was added DMAP (366 mg, 2.996 mmol, 0.1 equiv). The resulting mixture was stirred at 115° C. for 15 h under nitrogen atmosphere. The resulting mixture was concentrated under reduced pressure. The residue was dissolved in EA and washed with aqueous KHSO₄ (5%). The organic layers were dried over Na₂SO₄, filtered and concentrated under vacuum. This resulted in 4-(tert-butoxy)-4-oxobutanoic acid (4.00 g, crude) as a white solid.

### (2R,3R,11bR)-9,10-dimethoxy-3-(2-methylpropyl)-1H,2H,3H,4H,6H,7H,11bH-pyrido[2,1-a]isoquinolin-2-yl 1-tert-butyl butanedioate

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (600 mg, 1.878 mmol, 1 equiv), EDCl (1080 mg, 5.635 mmol, 3 equiv) and 4-(tert-butoxy)-4-oxobutanoic acid (579 mg, 3.325 mmol, 1.77 equiv) in DCM (6 mL) was added DMAP (241 mg, 1.972 mmol, 1 equiv) at room temperature. The resulting mixture was stirred at room temperature for 15 h. The resulting mixture was concentrated under reduced pressure. The residue was purified by silica gel column chromatography, eluted with PE/EA (3/1) to afford (2R,3R,11bR)-9,10-dimethoxy-3-(2-methylpropyl)-1H,2H,3H,4H,6H,7H,11bH-pyrido[2,1-a]isoquinolin-2-yl 1-tert-butyl butanedioate (500 mg 55.97%) as a light yellow solid.

### 4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid

To a stirred mixture of (2R,3R,11bR)-9,10-dimethoxy-3-(2-methylpropyl)-1H,2H,3H,4H,6H,7H,11bH-pyrido[2,1-a]isoquinolin-2-yl 1-tert-butyl butanedioate (500 mg, 1.051 mmol, 1 equiv) in DCM (5 mL) was added TFA (5 mL) at 0° C. The resulting mixture was stirred at room temperature for 24 h. The resulting mixture was concentrated under vacuum. This resulted in 4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid (390 mg crude) as a light yellow solid.

### [(2R,3R,11bR)-9,10-dimethoxy-3-(2-methylpropyl)-1H,2H,3H,4H,6H,7H,11bH-pyrido[2,1-a]isoquinolin-2-yl]butanedioate

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (475 mg, 0.933 mmol, 1.60 equiv) and 4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid (390 mg, 1.496 mmol, 1.00 equiv) in DCM (5 mL) was added DMAP (114 mg, 0.933 mmol, 1.00 equiv) and EDCl (536 mg, 2.799 mmol, 3.00 equiv) at room temperature. The resulting mixture was stirred at room temperature for 15 h. The resulting mixture was concentrated under vacuum. The residue was purified by Prep-HPLC with MeCN/water (3/1) to afford bis[(2R,3R,11bR)-9,10-dimethoxy-3-(2-methylpropyl)-1H,2H,3H,4H,6H,7H,11bH-pyrido[2,1-a]isoquinolin-2-yl]butanedioate (161.8 mg, 15.07%) as a white solid.

LCMS (ESI): [M+H]⁺: 721.5

¹H NMR (300 MHz, Methanol-d₄) δ 6.73-6.70 (m, 4H), 4.86-4.69 (m, 2H), 3.80-3.78 (m, 12H), 3.33-3.31 (m, 1H), 3.12-3.04 (m, 6H), 2.76-2.63 (m, 8H), 2.53-2.48 (m, 2H), 2.17-2.09 (m, 2H), 2.04-1.97 (m, 2H), 1.81-1.69 (m, 2H), 1.68-1.51 (m, 2H), 1.47-1.30 (m, 3H), 1.09-1.04 (m, 2H), 0.95-0.92 (m, 12H).

### Example 18: bis[(2R,3R,11bR)-9,10-dimethoxy-3-(2-methylpropyl)-1H,2H,3H,4H,6H,7H,11bH-pyrido[2,1-a]isoquinolin-2-yl] (2E)-but-2-enedioate

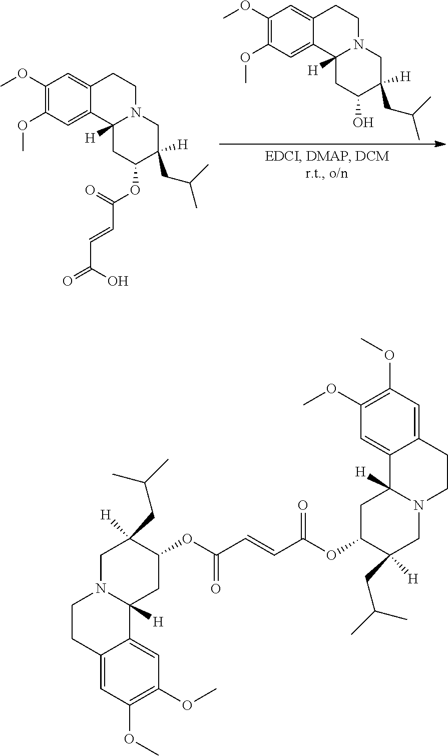 [SMILES: COc1cc2c(cc1OC)[C@H]1C[C@@H](OC(=O)/C=C/C(=O)O)[C@H](CC(C)C)CN1CC2 ; COc1cc2c(cc1OC)[C@H]1C[C@@H](O)[C@H](CC(C)C)CN1CC2 ; COc1cc2c(cc1OC)[C@H]1C[C@@H](OC(=O)/C=C/C(=O)O[C@@H]3C[C@@H]4c5cc(OC)c(OC)cc5CCN4C[C@H]3CC(C)C)[C@H](CC(C)C)CN1CC2]

To a stirred solution of (2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid (430 mg, 1.030 mmol, 1 equiv) and (2R,3R,11bR)-9,10-dimethoxy-3-(2-methylpropyl)-1H,2H,3H,4H,6H,7H,11bH-pyrido[2,1-a]isoquinolin-2-ol (526 mg, 1.648 mmol, 1.6 equiv) in DCM (10 mL) was added DMAP (126 mg, 1.030 mmol, 1 equiv) and EDCl (592 mg, 3.090 mmol, 3 equiv). The mixture was stirred at room temperature overnight. The residue was purified by Prep-HPLC with MeCN/H₂O=75/25 to afford bis[(2R,3R,11bR)-9,10-dimethoxy-3-(2-methylpropyl)-1H,2H,3H,4H,6H,7H,11bH-pyrido[2,1-a]isoquinolin-2-yl] (2E)-but-2-enedioate (226.8 mg, 30.63%) as a white solid.

LCMS (ESI): [M+H]⁺: 719.4

¹H NMR (400 MHz, Methanol-d₄) δ 6.91 (s, 2H), 6.75 (s, 2H), 6.71 (s, 2H), 4.86-4.78 (m, 2H), 3.82-3.79 (m, 12H), 3.16-3.06 (m, 7H), 2.75-2.67 (m, 4H), 2.69-2.45 (m, 2H), 2.28-2.12 (m, 2H), 2.11-2.00 (m, 2H), 1.79-1.61 (m, 2H), 1.57-1.51 (m, 2H), 1.36-1.25 (m, 3H), 1.16-1.12 (m, 2H), 0.96-0.93 (m, 12H).

### Example 19: bis[(2R,3R,11bR)-9,10-dimethoxy-3-(2-methylpropyl)-1H,2H,3H,4H,6H,7H,11bH-pyrido[2,1-a]isoquinolin-2-yl] (2Z)-but-2-enedioate

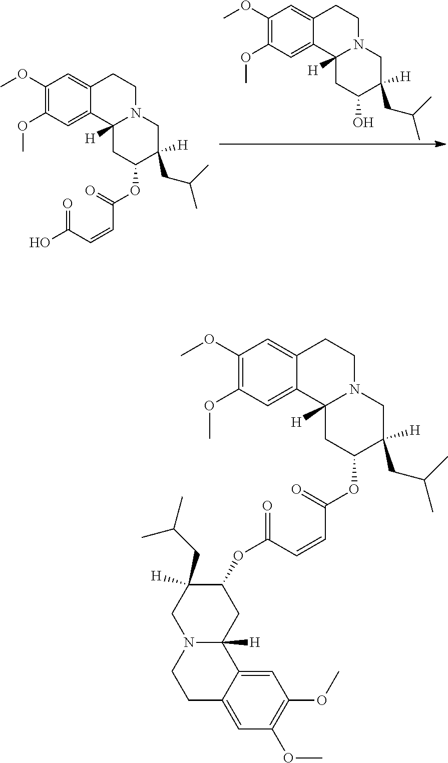 [SMILES: COc1cc2c(cc1OC)[C@H]1C[C@@H](OC(=O)/C=C\C(=O)O)[C@H](CC(C)C)CN1CC2 ; COc1cc2c(cc1OC)[C@H]1C[C@@H](O)[C@H](CC(C)C)CN1CC2 ; COc1cc2c(cc1OC)[C@H]1C[C@@H](OC(=O)/C=C\C(=O)O[C@@H]3C[C@@H]4c5cc(OC)c(OC)cc5CCN4C[C@H]3CC(C)C)[C@H](CC(C)C)CN1CC2]

To a stirred mixture of (2R,3R,11bR)-9,10-dimethoxy-3-(2-methylpropyl)-1H,2H,3H,4H,6H,7H,11bH-pyrido[2,1-a]isoquinolin-2-ol (275 mg, 0.861 mmol, 1.5 equiv) and (2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid (240 mg, 0.575 mmol, 1 equiv) in DCM (4 ml) was added EDCl (331 mg, 1.727 mmol, 3.00 equiv) and DMAP (70 mg, 0.573 mmol, 1 equiv) at room temperature. The reaction was stirred at room temperature for 14 h. The reaction was concentrated. The residue was purified by Prep-HPLC with MeCN/water (7/3). This resulted in bis[(2R,3R,11bR)-9,10-dimethoxy-3-(2-methylpropyl)-1H,2H,3H,4H,6H,7H,11bH-pyrido[2,1-a]isoquinolin-2-yl] (2Z)-but-2-enedioate (160.1 mg) as a light yellow solid.

LCMS (ESI): [M+H]⁺: 719.5,

¹H NMR (300 MHz, Methanol-d₄) δ 6.90 (s, 2H), 6.74 (s, 2H), 6.70 (s, 2H), 4.92-4.85 (m, 2H), 3.83-3.78 (m, 12H), 3.33-3.31 (m, 2H), 3.16-3.03 (m, 6H), 2.75-2.71 (m, 4H), 2.55-2.51 (m, 2H), 2.23-2.05 (m, 4H), 1.69-1.51 (m, 4H), 1.36-1.23 (m, 2H), 1.19-1.01 (m, 2H), 1.00-0.89 (m, 12H).

### Example 20: Bis[(2R,3R,11bR)-9,10-dimethoxy-3-(2-methylpropyl)-1H,2H,3H,4H,6H,7H,11bH-pyrido[2,1-a]isoquinolin-2-yl] oxalate

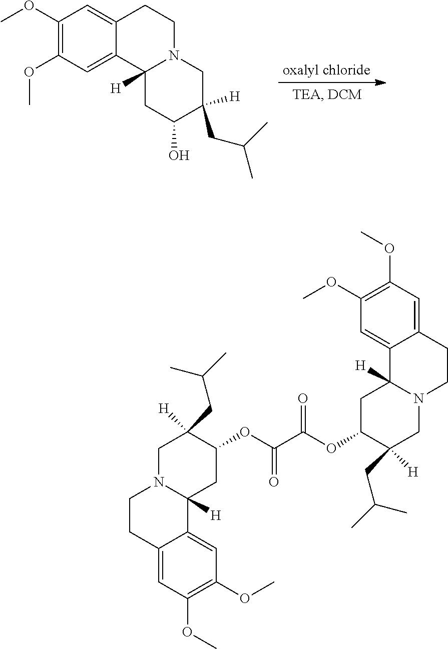 [SMILES: COc1cc2c(cc1OC)[C@H]1C[C@@H](O)[C@H](CC(C)C)CN1CC2 ; COc1cc2c(cc1OC)[C@H]1C[C@@H](OC(=O)C(=O)O[C@@H]3C[C@@H]4c5cc(OC)c(OC)cc5CCN4C[C@H]3CC(C)C)[C@H](CC(C)C)CN1CC2]

To a mixture of (2R,3R,11bR)-9,10-dimethoxy-3-(2-methylpropyl)-1H,2H,3H,4H,6H,7H,11bH-pyrido[2,1-a]isoquinolin-2-ol (400 mg, 1.252 mmol, 1 equiv) in DCM (3 mL) was added TEA (126.7 mg, 1.252 mmol, 1 equiv) and oxalyl chloride (1.9 mL, 2 mol/L, 3 equiv) at 0° C. The resulting solution was stirred at room temperature for 1 h. The residue was applied onto a silica gel column with ethyl acetate/petroleum ether (1/1). The crude product (160 mg) was purified by Prep-HPLC with MeCN/H₂O=60/40. This resulted in 127.5 mg (14.70%) of bis[(2R,3R,11bR)-9,10-dimethoxy-3-(2-methylpropyl)-1H,2H,3H,4H,6H,7H,11bH-pyrido[2,1-a]isoquinolin-2-yl] oxalate as a white solid.

LCMS (ESI): [M+H]⁺: 693.5

¹H NMR (400 MHz, Methanol-d₄) δ 6.76 (s, 2H), 6.72 (s, 2H), 4.92-4.87 (m, 2H), 3.81-3.80 (m, 12H), 3.34-3.32 (m, 2H), 3.16-3.01 (m, 6H), 2.79-2.70 (m, 4H), 2.69-2.49 (m, 2H), 2.25-2.19 (m, 2H), 2.29-2.09 (m, 2H), 1.73-1.71 (m, 2H), 1.71-1.59 (m, 2H), 1.38-1.32 (m, 2H), 1.73-1.13 (m, 2H), 0.68-0.93 (m, 12H).

### Example 21: (2R,3R,11bR)-9,10-dimethoxy-3-(2-methylpropyl)-1H,2H,3H,4H,6H,7H,11bH-pyrido[2,1-a]isoquinolin-2-yl N-ethylcarbamate

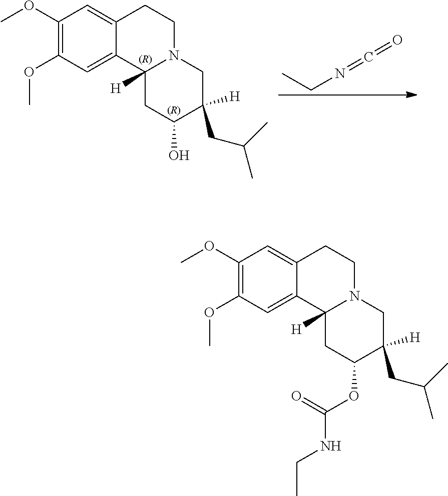

To a stirred mixture of (2R,3R,11bR)-9,10-dimethoxy-3-(2-methylpropyl)-1H,2H,3H, 4H,6H,7H,11bH-pyrido[2,1-a]isoquinolin-2-ol (250 mg, 0.783 mmol, 1 equiv) in 1,4-dioxane (3 mL) was added isocyanatoethane (835 mg, 11.747 mmol, 15.01 equiv) at room temperature under nitrogen atmosphere. The resulting mixture was stirred at 120° C. for 15 h under nitrogen atmosphere. The resulting mixture was concentrated under reduced pressure. The residue was purified by Prep-HPLC with MeCN/water (3/1) to afford (2R,3R,11bR)-9,10-dimethoxy-3-(2-methylpropyl)-1H,2H,3H,4H,6H,7H,11bH-pyrido[2,1-a]isoquinolin-2-yl N-ethylcarbamate (148.3 mg, 48.04%) as a light yellow solid.

LCMS (ESI): [M+H]⁺: 391.3

¹H NMR (400 MHz, Methanol-d₄) δ 6.76 (s, 1H), 6.70 (s, 1H), 4.88-4.50 (m, 1H), 3.82-3.80 (m, 6H), 3.33-3.21 (m, 1H), 3.21-3.04 (m, 5H), 2.73-2.68 (m, 2H), 2.54-2.51 (m, 1H), 2.16-2.10 (m, 1H), 1.97-1.88 (m, 1H), 1.72-1.63 (m, 1H), 1.48-1.38 (m, 2H), 1.16-1.08 (m, 4H), 0.96-0.91 (m, 6H).

### Example 22: (2R,3R,11bR)-9,10-dimethoxy-3-(2-methylpropyl)-1H,2H,3H,4H,6H,7H,11bH-pyrido[2,1-a]isoquinolin-2-yl N-isopropylcarbamate

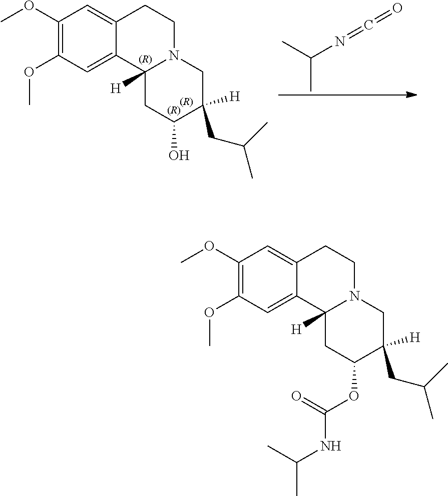

To a mixture of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) in dioxane (2 mL) was added 2-isocyanatopropane (1.9 g, 23.478 mmol, 15 equiv). The resulting solution was stirred at 120° C. overnight under nitrogen. The crude product was purified by Prep-HPLC with Water/ACN (20/80). This resulted in 205.6 mg (32.18%) of (2R,3R,11bR)-9,10-dimethoxy-3-(2-methylpropyl)-1H,2H,3H,4H,6H,7H,11bH-pyrido[2,1-a]isoquinolin-2-yl N-isopropylcarbamate as a white solid.

LCMS (ESI): [M+H]⁺: 405.4

¹H NMR (300 MHz, Methanol-d₄) δ 6.75 (s, 1H), 6.68 (s, 1H), 4.49-4.48 (m, 1H), 3.78-3.72 (m, 7H), 3.30-3.22 (m, 1H), 3.10-3.00 (m, 3H), 2.71-2.64 (m, 2H), 2.52-2.46 (m, 1H), 2.15-2.07 (m, 1H), 2.01-1.82 (m, 1H), 1.81-1.58 (m, 1H), 1.53-1.31 (m, 2H), 1.06-1.07 (m, 7H), 0.94-0.89 (m, 6H).

### Example 23: (2R,3R,11bR)-9,10-dimethoxy-3-(2-methylpropyl)-1H,2H,3H,4H,6H,7H,11bH-pyrido[2,1-a]isoquinolin-2-yl N-(2-methylpropyl)carbamate

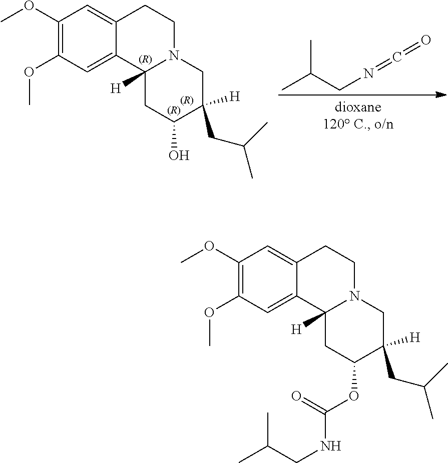

To a stirred solution of (2R,3R,11bR)-9,10-dimethoxy-3-(2-methylpropyl)-1H,2H,3H,4H,6H,7H,11bH-pyrido[2,1-a]isoquinolin-2-ol (500 mg, 1.565 mmol, 1 equiv) in dioxane (2 mL) was added 1-isocyanato-2-methylpropane (2.3 g, 23.504 mmol, 15 equiv). The resulting mixture was stirred at 120° C. overnight. The crude product (200 mg) was purified by Prep-HPLC with MeCN/H₂=(65/35) to afford 85.8 mg (13.10%) of (2R,3R,11bR)-9,10-dimethoxy-3-(2-methylpropyl)-1H,2H,3H,4H,6H,7H,11bH-pyrido[2,1-a]isoquinolin-2-yl N-(2-methylpropyl)carbamate as a pink solid.

LCMS (ESI): [M+H]⁺: 419.4

¹H NMR (400 MHz, Methanol-d₄) δ 6.77 (s, 1H), 6.71 (s, 1H), 4.52-4.51 (m, 1H), 3.81 (d, J=2.4 Hz, 6H), 3.28-3.25 (m, 1H), 2.90-2.73 (m, 5H), 2.73-2.69 (m, 2H), 2.69-2.52 (m, 1H), 2.06-2.11 (m, 1H), 2.08-1.90 (m, 1H), 1.89-1.71 (m, 2H), 1.58-1.41 (m, 2H), 1.17-1.09 (m, 1H), 0.94-0.92 (m, 12H).

### Example 24: [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methylpropanoate

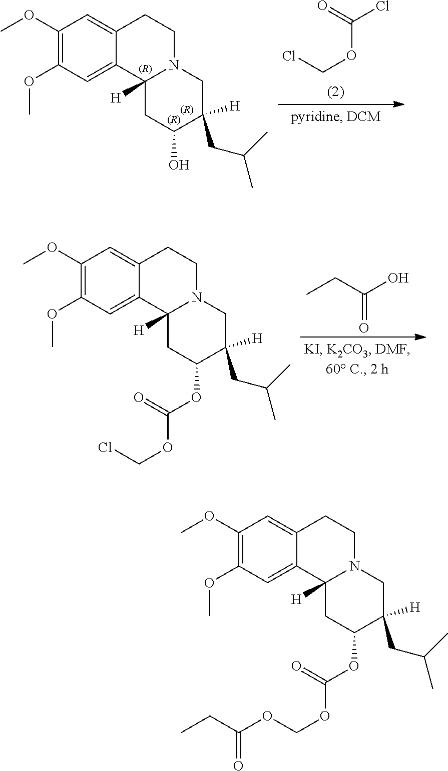

### (2R,3R,11bR)-9,10-dimethoxy-3-(2-methylpropyl)-1H,2H,3H,4H,6H,7H,11bH-pyrido[2,1-a]isoquinolin-2-yl chloromethyl carbonate

To a mixture of (2R,3R,11bR)-9,10-dimethoxy-3-(2-methylpropyl)-1H,2H,3H,4H,6H,7H,11bH-pyrido[2,1-a]isoquinolin-2-ol (1 g, 3.130 mmol, 1 equiv) in DCM (10 mL) was added pyridine (495 mg, 6.261 mmol, 2 equiv). Then chloromethyl chloroformate (1.2 g, 9.384 mmol, 3 equiv) was added. The resulting mixture was stirred at room temperature for 30 min. The resulting mixture was quenched with water and extracted with 3×10 mL of dichloromethane. The organic layers were combined and concentrated under vacuum. The residue was applied onto a silica gel column with dichloromethane/methanol (20/1). This resulted in 1.1 g (85.31%) of (2R,3R,11bR)-9,10-dimethoxy-3-(2-methylpropyl)-1H,2H,3H,4H,6H,7H,11bH-pyrido[2,1-a]isoquinolin-2-yl chloromethyl carbonate as a light yellow solid.

LCMS (ESI): [M+H]⁺: 412.2

### [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl propanoate

To a mixture of (2R,3R,11bR)-9,10-dimethoxy-3-(2-methylpropyl)-1H,2H,3H,4H,6H,7H,11bH-pyrido[2,1-a]isoquinolin-2-yl chloromethyl carbonate (400 mg, 0.971 mmol, 1 equiv) in DMF (3 mL) were added KI (80 mg, 0.486 mmol, 0.5 equiv), K₂CO₃ (134 mg, 0.971 mmol, 1 equiv) and propanoic acid (144 mg, 1.942 mmol, 2 equiv). The resulting solution was stirred at 60° C. for 2 h. The crude product was purified by Flash-Prep-HPLC with MeCN/H₂O=60/40. This resulted in 231.6 mg (53.05%) of [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl propanoate as a yellow semi-solid.

LCMS (ESI): [M+H]⁺: 450.2

¹H NMR (300 MHz, Methanol-d₄) δ 6.75 (s, 1H), 6.71 (s, 1H), 5.82-5.76 (m, 2H), 4.71-4.51 (m, 1H), 3.80 (s, 6H), 3.31-3.29 (m, 1H), 3.20-3.03 (m, 3H), 2.78-2.69 (m, 2H), 2.61-2.50 (m, 1H), 2.52-2.39 (m, 2H), 2.61-2.52 (m, 1H), 2.12-1.91 (m, 1H), 1.80-1.59 (m, 1H), 1.59-1.52 (m, 1H), 1.51-1.31 (m, 1H), 1.33-1.05 (m, 4H), 0.95-0.91 (m, 6H).

### Example 25: [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 2-methylpropanoate

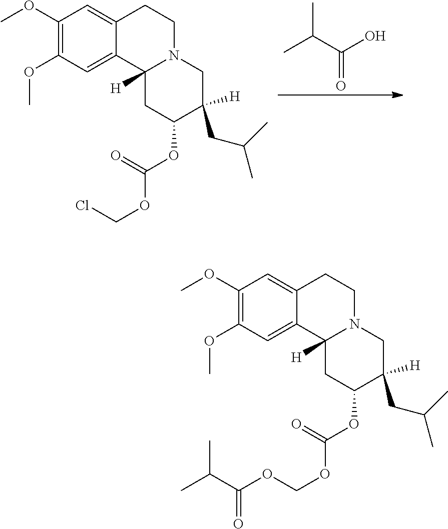

To a stirred mixture of (2R,3R,11bR)-9,10-dimethoxy-3-(2-methylpropyl)-1H,2H,3H, 4H,6H, 7H, 11bH-pyrido[2,1-a]isoquinolin-2-yl chloromethyl carbonate (350 mg, 0.850 mmol, 1 equiv) and K₂CO₃ (117 mg, 0.847 mmol, 1 equiv) in DMF (4 mL) was added KI (71 mg, 0.428 mmol, 0.5 equiv) and isobutyric acid (150 mg, 1.702 mmol, 2 equiv) at 60° C. The resulting mixture was stirred at 60° C. for 2 h. The resulting mixture was concentrated under vacuum. The residue was purified by Prep-HPLC with MeCN/water (7/1) to afford [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 2-methylpropanoate (219.6 mg, 55.75%) as a light yellow solid.

LCMS (ESI): [M+H]⁺: 464.2

¹H NMR (300 MHz, Methanol-d₄) δ 6.74 (s, 1H), 6.71 (s, 1H), 5.82-5.76 (m, 2H), 4.86-4.57 (m, 1H), 3.95-3.80 (m, 6H), 3.13-3.03 (m, 3H), 2.77-2.52 (m, 4H), 2.21-2.13 (m, 1H), 2.13-1.94 (m, 1H), 1.82-1.70 (s, 1H), 1.70-1.52 (m, 1H), 1.36-1.32 (m, 1H), 1.20-1.12 (m, 8H), 0.95-0.90 (m, 6H).

### Example 26: [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 3-methylbutanoate

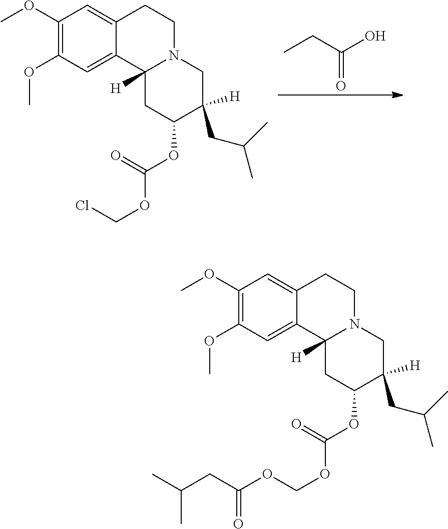

To a stirred mixture of (2R,3R,11bR)-9,10-dimethoxy-3-(2-methylpropyl)-1H,2H,3H,4H,6H,7H,11bH-pyrido[2,1-a]isoquinolin-2-yl chloromethyl carbonate (240 mg, 0.583 mmol, 1 equiv), K₂CO₃ (79 mg, 0.572 mmol, 1 equiv) and KI (48 mg, 0.289 mmol, 0.5 equiv) in DMF (4 mL) was added isovaleric acid (120 mg, 1.175 mmol, 2 equiv). The reaction was stirred at 60° C. for 2 h. The reaction was purified by Prep-HPLC with MeCN/water (7/3) to afford [([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 3-methylbutanoate (210.4 mg, 75.61%) as a yellow solid.

LCMS (ESI): [M+H]⁺: 478.1

¹H NMR (300 MHz, Methanol-d₄) δ 6.69 (s, 1H), 6.68 (s, 1H), 5.79-5.74 (m, 2H), 4.57-4.56 (m, 1H), 3.78-3.70 (m, 6H), 3.31-3.29 (m, 1H), 3.12-3.02 (m, 3H), 2.76-2.68 (m, 2H), 2.68-2.50 (m, 1H), 2.28-2.26 (m, 2H), 2.19-2.04 (m, 3H), 1.73-1.49 (m, 2H), 1.35-1.35 (m, 1H), 1.16-1.10 (m, 1H), 0.98-0.89 (m, 12H).

Biological Results

Metabolism of compound(s) to release (+)-α-DTHBZ was determined by the half-life (t_{1/2}) and intrinsic clearance (CIᵢₙₜ) in human plasma (PS) and liver microsomes (LM) using tandem mass spectrometry (LC-MS/MS).

| | Human LM | | |
|---|---|---|---|
| | | CLᵢₙₜ | |
| Example | | (μL/min/mg | Human PS |
| No. | t_{1/2} (min) | protein) | t_{1/2} (min) |
| 1 | 414.63 | 3.24 | ∞ |
| 2 | >879.68 | <0.79 | ∞ |
| 3 | ∞ | 0.00 | 762.94 |
| 4 | >203.65 | <3.40 | 3301.52 |
| 5 | 233.53 | 3.16 | ∞ |
| 7 | 10.58 | 65.80 | >811.70 |
| 8 | 7.07 | 98.05 | >709.17 |
| 10 | 6.67 | 103.86 | >226167.89 |
| 11 | 176.73 | 4.24 | 13.52 |
| 13 | 345.55 | 2.17 | 142.95 |
| 14 | 249.45 | 2.91 | 12.01 |
| 18 | 1248.29 | 1.11 | >662.74 |
| 21 | 483.10 | 1.67 | >20441.58 |
| 22 | 138.77 | 5.15 | >3928.55 |
| 23 | >175.49 | <3.95 | 26253.18 |
| 24* | NV | NV | 85.38 |
| 25* | NV | NV | 118.67 |
| 6 | >1974.53 | <0.35 | >2684.88 |
| 12 | 691.69 | 1.42 | 14.27 |
| 16 | >552.88 | <1.25 | ∞ |
| 17 | 456.24 | 1.59 | 2213.92 |
| 20 | 191.87 | 3.83 | 80.84 |
| 26 | 0.50 | 1381.49 | 83.35 |
| 9 | 42.48 | 16.32 | ∞ |
| 15 | >8564.63 | <0.08 | >3219.53 |
| 19 | >821.24 | 0.84 | 8017.96 |

*Extremely unstable in Human liver microsomes, the compound could not be detected in sample of 0.5 min time point.

Metabolism of compound(s) to release (+)-α-DTHBZ was determined by the half-life (t_{1/2}) and intrinsic clearance (CIᵢₙₜ) in human liver microsomes (LM) using tandem mass spectrometry (LC-MS/MS).

| | LM stability | |
|---|---|---|
| | assay | |
| | Human | |
| | | Clᵢₙₜ |
| Example | t_{1/2} | (μL/min/mg |
| No. | (min) | protein) |
| 2 | ∞ | 0.00 |
| 5 | 3410.18 | 1.04 |
| 6 | 802.07 | 1.67 |
| 7 | 0.75 | 922.00 |
| 9 | 1.13 | 613.24 |
| 12 | 534.68 | 2.38 |
| 13 | 3068.54 | 0.35 |
| 15 | ∞ | 0.00 |
| 16 | 4.65 | 149.50 |
| 17 | 9.71 | 71.48 |
| 23 | 3.34 | 207.87 |

Metabolism of compound(s) to release (+)-α-DTHBZ was determined by the half-life (t_{1/2}) and intrinsic clearance (CIᵢₙₜ) in human cryopreserved hepatocytes using tandem mass spectrometry (LC-MS/MS).

| | Heps. stability | |
|---|---|---|
| | assay | |
| | Human | |
| | | Clᵢₙₜ |
| Example | t_{1/2} | (μL/min/mg |
| No. | (min) | protein) |
| 2 | ∞ | 0.00 |
| 5 | 27.38 | 50.66 |
| 6 | ∞ | 0.00 |
| 7 | 4.92 | 281.78 |
| 9 | 11.47 | 120.96 |
| 12 | 221.23 | 6.27 |
| 13 | 293.53 | 4.75 |
| 15 | 83.68 | 16.57 |
| 16 | 61.66 | 22.59 |
| 17 | 81.93 | 16.95 |
| 23 | 38.96 | 35.57 |

Kinetic solubility was determined by suspending compound at 300 μM at pH 1.2 and 7.4 in PBS buffer. The suspension was equilibrated by shaking at 25° C. at 1100 RPM for 2 hours and then pH was measured following filtration. The filtrate was then diluted by an appropriate factor (e.g. 100-fold). Quantitation was done by LC-MS/MS with reference to a standard solution.

| | Kinetic | |
|---|---|---|
| | Solubility | |
| Example | pH = 1.2 | pH = 7.4 |
| No. | (300 μM) | (300 μM) |
| 2 | 281.25 | 294.79 |
| 5 | 276.13 | 306.09 |
| 6 | 276.47 | 294.26 |
| 7 | 310.51 | 5.84 |
| 9 | 292.34 | 0.0018 |
| 12 | 319.98 | 306.12 |
| 13 | 302.86 | 300.00 |
| 15 | 316.81 | 0.060 |
| 16 | 140.92 | 0.042 |
| 17 | 308.49 | 0.0019 |
| 23 | 312.09 | 91.15 |

The intestinal permeability of compound(s) was evaluated in Caco-2 cells by assessing transport across the cell monolayer in the apical to basolateral (A-B) direction. Active efflux was also assessed by measuring transport in the basolateral to apical (BA) direction which enables evaluation of the efflux ratio. Analysis of transport is quantified by tandem mass spectrometry (LC-MS/MS).

| | Permeability in Caco-2 Cell | | |
|---|---|---|---|
| Example | P_{app (A-B)} | P_{app (B-A)} | Efflux |
| No. | (10⁻⁶, cm/s) | (10⁻⁶, cm/s) | Ratio |
| 2 | 8.67 | 22.00 | 2.54 |
| 5 | 22.40 | 21.98 | 0.98 |
| 6 | 0.68 | 17.06 | 25.05 |
| 7 | 1.78 | 1.50 | 0.84 |
| 9 | 0.2020 | 0.1910 | 0.95 |
| 12 | 0.13 | 11.32 | 85.42 |
| 13 | <0.01 | 4.02 | >401.86 |
| 15 | <0.01 | <0.01 | NC |
| 16 | <0.02 | <0.01 | NC |
| 17 | <0.02 | 0.0333 | >1.93 |
| 23 | 14.17 | 9.54 | 0.67 |

What is claimed is:

1. A compound of Formula (I) or Formula (II): 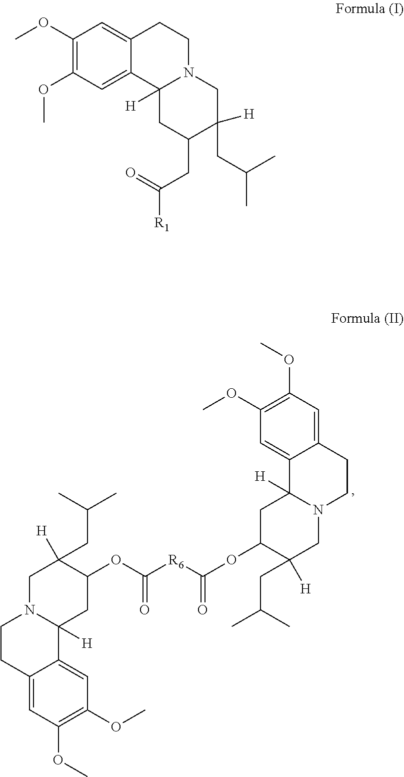
wherein:
R₁ is —C(O)R₂, —(C₁-C₆alkyl)-C(O)R₂, —(C₂-C₄alkenyl)-C(O)R₂, —NH—(C₁-C₄alkyl), —O—CH₂—O—C(O)—(C₁-C₄alkyl), or 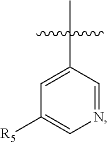
R₂ is —OR₃ or —NH—CH(—C(O)OH)—R₄,
R₃ is —H or —C₁-C₄alkyl,
R₄ is —C₁-C₄alkyl, optionally substituted by a phenyl group,
R₅ is —C(O)OR₃, and
R₆ is a bond, —C₁-C₈alkyl-, —C₂-C₄alkenyl-, or 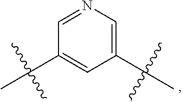 [SMILES: *c1cncc(*)c1]
or a pharmaceutically acceptable salt, ester, hydrate, or solvate thereof.

2. The compound of claim 1, wherein the compound of Formula (I) has the following structure:

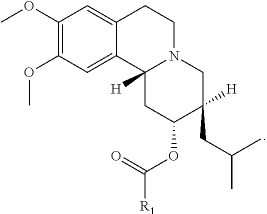

3. The compound of claim 1, wherein for the compound of Formula (I):
R₁ is —C(O)R₂, —CH₂CH₂—C(O)R₂, —(CH₂)₆—C(O)R₂, —CH═CH—C(O)R₂, —NH—CH₂CH₃, —NH—CH(CH₃)₂, —NH—CH₂CH(CH₃)₂, —O—CH₂—O—C(O)—CH₂CH₃, —O—CH₂—O—C(O)—CH(CH₃)₂, —O—CH₂—O—C(O)—CH₂CH(CH₃)₂, or 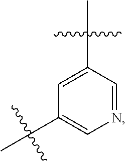 [SMILES: *c1cncc(*)c1]
R₂ is —OR₃ or —NH—CH(—C(O)OH)—R₄,
R₃ is —H or —CH(CH₃)₂,
R₄ is —CH(CH₃)₂ or —CH₂(phenyl), and
R₅ is —C(O)OR₃.

4. The compound of claim 1, wherein the compound of Formula (I) is selected from the group consisting of:
(2S,3S,11bS)-9,10-dimethoxy-3-(2-methylpropyl)-1H,2H,3H,4H,6H,7H,11bH-pyrido[2,1-a]isoquinolin-2-yl hydrogen oxalate;
4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobutanoic acid;
(2E)-4-[[(2S,3S,11bS)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid;
(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enoic acid;
8-[[(2R,3R,11bR)-9,10-dimethoxy-3-(2-methylpropyl)-1H,2H,3H,4H,6H,7H,11bH-pyrido[2,1-a]isoquinolin-2-yl]oxy]-8-oxooctanoic acid;
5-([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)pyridine-3-carboxylic acid;
(2R,3R,11bR)-9,10-dimethoxy-3-(2-methylpropyl)-1H,2H,3H,4H,6H,7H,11bH-pyrido[2,1-a]isoquinolin-2-yl 1-isopropyl butanedioate;
(2R,3R,11bR)-9,10-dimethoxy-3-(2-methylpropyl)-1H,2H,3H,4H,6H,7H,11bH-pyrido[2,1-a]isoquinolin-2-yl 1-isopropyl (2E)-but-2-enedioate;
3-((2R,3R,11bR)-3-isobutyl-9,10-dimethoxy-1,3,4,6,7,11b-hexahydro-2H-pyrido[2,1-a]isoquinolin-2-yl) 5-isopropyl pyridine-3,5-dicarboxylate;
(2R,3R,11bR)-9,10-dimethoxy-3-(2-methylpropyl)-1H,2H,3H,4H,6H,7H,11bH-pyrido[2,1-a]isoquinolin-2-yl 1-isopropyl (2Z)-but-2-enedioate;
(2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid;
(2S)-2-[(2E)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid;
(2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-methylbutanoic acid;
(2S)-2-[(2Z)-4-[[(2R,3R,11bR)-9,10-dimethoxy-3-(2-methylpropyl)-1H,2H,3H,4H,6H,7H,11bH-pyrido[2,1-a]isoquinolin-2-yl]oxy]-4-oxobut-2-enamido]-3-phenylpropanoic acid;
(2R,3R,11bR)-9,10-dimethoxy-3-(2-methylpropyl)-1H,2H,3H,4H,6H,7H,11bH-pyrido[2,1-a]isoquinolin-2-yl N-ethylcarbamate;
(2R,3R,11bR)-9,10-dimethoxy-3-(2-methylpropyl)-1H,2H,3H,4H,6H,7H,11bH-pyrido[2,1-a]isoquinolin-2-yl N-isopropyl carbamate;
(2R,3R,11bR)-9,10-dimethoxy-3-(2-methylpropyl)-1H,2H,3H,4H,6H,7H,11bH-pyrido[2,1-a]isoquinolin-2-yl N-(2-methylpropyl)carbamate;
[([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl propanoate;
[([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 2-methylpropanoate; and
[([[(2R,3R,11bR)-9,10-dimethoxy-3-(2-methylpropyl)-1H,2H,3H,4H,6H,7H,11bH-pyrido[2,1-a]isoquinolin-2-yl]oxy]carbonyl)oxy]methyl 3-methylbutanoate.

5. The compound of claim 1, wherein the compound of Formula (II) has the following structure:

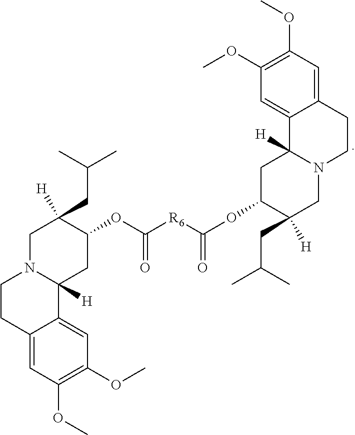

6. The compound of claim 1, wherein for the compound of Formula (II):
R₆ is a bond, —CH₂CH₂—, —(CH₂)₆—, —CH═CH—, or

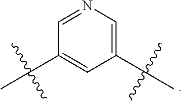 [SMILES: *c1cncc(*)c1]

7. The compound of claim 1, wherein the compound of Formula (II) is selected from the group consisting of:
3,5-bis[(2R,3R,11bR)-9,10-dimethoxy-3-(2-methylpropyl)-1H,2H,3H,4H,6H,7H,11bH-pyrido[2,1-a]isoquinolin-2-yl] pyridine-3,5-dicarboxylate;
bis[(2R,3R,11bR)-9,10-dimethoxy-3-(2-methylpropyl)-1H,2H,3H,4H,6H,7H,11bH-pyrido[2,1-a]isoquinolin-2-yl] octanedioate;
[(2R,3R,11bR)-9,10-dimethoxy-3-(2-methylpropyl)-1H,2H,3H,4H,6H,7H,11bH-pyrido[2,1-a]isoquinolin-2-yl] butanedioate;
bis[(2R,3R,11bR)-9,10-dimethoxy-3-(2-methylpropyl)-1H,2H,3H,4H,6H,7H,11bH-pyrido[2,1-a]isoquinolin-2-yl] (2E)-but-2-enedioate;
bis[(2R,3R,11bR)-9,10-dimethoxy-3-(2-methylpropyl)-1H,2H,3H,4H,6H,7H,11bH-pyrido[2,1-a]isoquinolin-2-yl] (2Z)-but-2-enedioate; and
bis[(2R,3R,11bR)-9,10-dimethoxy-3-(2-methylpropyl)-1H,2H,3H,4H,6H,7H,11bH-pyrido[2,1-a]isoquinolin-2-yl] oxalate.

8. A pharmaceutical composition comprising, consisting essentially of, or consisting of a therapeutically effective amount of a compound of claim 1, and at least one pharmaceutically acceptable excipient.

9. The pharmaceutical composition of claim 8, wherein the pharmaceutical composition is a unit dosage form.

10. The pharmaceutical composition of claim 9, wherein the unit dosage form is a solid oral dosage form.

11. The pharmaceutical composition of claim 8, wherein the pharmaceutical composition disintegrates in oral fluid and is substantially absorbed in the oral mucosa.

12. A method of administering the pharmaceutical composition of claim 8 to a mammal, comprising:
a) providing the pharmaceutical composition; and
b) administrating the pharmaceutical composition to the mammal.

13. The method of claim 12, wherein the pharmaceutical composition provides a Cₘₐₓ of between about 75% to about 125% that of a Xenazine® formulation wherein the latter includes at least 80% more 2R,3R,11bR-dihydrotetrabenazine by weight.

14. The method of claim 12, wherein the pharmaceutical composition is administered to treat a VMAT2-mediated disorder.

15. The method of claim 14, wherein the VMAT2-mediated disorder is selected from the group consisting of chronic hyperkinetic disorder, Huntington's disease, hemiballismus, senile chorea, tic disorders, tardive dyskinesia, levodopa-induced dyskinesia, dystonia, Tourette's syndrome, depression, cancer, rheumatoid arthritis, psychosis, multiple sclerosis, asthma, Parkinson's disease, drug addiction, environmental neurotoxin-mediated neurodegeneration, bipolar disorder, and schizophrenia.

16. The method of claim 15, wherein the VMAT2-mediated disorder is a chronic hyperkinetic disorder.

17. The method of claim 12, wherein the mammal is a human.

18. A method of treating a VMAT2-mediated disorder to a mammal in need thereof, comprising administering the pharmaceutical composition of claim 8 to the mammal.

19. The method of claim 18, wherein the VMAT2-mediated disorder is selected from the group consisting of chronic hyperkinetic disorder, Huntington's disease, hemiballismus, senile chorea, tic disorders, tardive dyskinesia, levodopa-induced dyskinesia, dystonia, Tourette's syndrome, depression, cancer, rheumatoid arthritis, psychosis, multiple sclerosis, asthma, Parkinson's disease, drug addiction, environmental neurotoxin-mediated neurodegeneration, bipolar disorder, and schizophrenia.

20. The method of claim 18, wherein the mammal is a human.
